(19) United States
(12) Patent Application Publication
(10) Pub. No.: US 2022/0288010 A1
(43) Pub. Date: Sep. 15, 2022

(54) METHODS FOR REDUCING REWARDING EFFECTS OF MORPHINE WITHOUT AFFECTING ITS ANALGESIC EFFECTS

(71) Applicant: The Board of Trustees of the Leland Stanford Junior University, Stanford, CA (US)

(72) Inventors: Francis Lee, New York, NY (US); Anjali M. Rajadhyaksha, New York, NY (US); Arlene Martinez-Rivera, Bronx, NY (US)

(73) Assignee: The Board of Trustees of the Leland Stanford Junior University, Stanford, CA (US)

(21) Appl. No.: 17/702,578

(22) Filed: Mar. 23, 2022

Related U.S. Application Data
(63) Continuation of application No. PCT/US2020/052832, filed on Sep. 25, 2020.
(60) Provisional application No. 62/906,536, filed on Sep. 26, 2019.

(51) Int. Cl. A61K 31/343; A61P 25/04; A61K 31/27; A61K 45/06

(52) U.S. Cl. CPC A61K 31/343; A61P 25/04; A61K 31/27; A61K 45/06

(57) ABSTRACT

The present disclosure provides methods and compositions for inhibiting monoacylglycerol lipase (MAGL) in order to attenuate the rewarding effects of and delay tolerance to the analgesic properties of opioids such as morphine without reducing their analgesic effects.

### CROSS REFERENCE TO RELATED APPLICATIONS

The present application is a continuation of International Application No. PCT/US2020/052832, filed on Sep. 25, 2020, which claims priority to U.S. Provisional Pat. Appl. No. 62/906,536, filed on Sep. 26, 2019, the disclosures of which are hereby incorporated by reference in their entirety for all purposes.

### BACKGROUND

Opioids are potent analgesics commonly used in the clinic; however, they are also rewarding and their long-term use can promote dependence that often transitions to addiction. In addition, prolonged use of opioids can lead to tolerance effects, where increasing amounts of the opioid are required in order to achieve the same therapeutic analgesic effect. Morphine, for example, is a commonly used opioid prescription analgesic that can cause dependence because of its rewarding properties. There is a critical need for the development of novel strategies that can serve as adjuvants for use with opioid medications such as morphine that can attenuate the rewarding aspects of opioids and delay tolerance, while maintaining their analgesic properties.

Monoacylglycerol lipase, or MAG lipase or MAGL, is a serine hydrolase that participates in the hydrolysis of monoglycerides to form glycerol and fatty acids. MAGL is involved, e.g., in the hydrolysis of the endocannabinoid, 2-arachidonoylglycerol (2-AG), in a reaction giving rise to arachidonic acid. 2-AG is the most prevalent endogenous cannabinoid ligand in the brain, and is an agonist of the CB1 and CB2 receptors and the primary ligand for the CB2 receptor.

Currently no pharmacological interventions exist to dissociate the analgesic and rewarding properties of morphine or other opioids, which would be useful to reduce the rewarding effects of these agents while maintaining their analgesic properties. The present invention satisfies this need and provides other advantages as well.

### BRIEF SUMMARY

In one aspect, the present disclosure provides a method of attenuating the rewarding effect of an opioid in a subject, the method comprising administering a therapeutically effective amount of a monoacylglycerol lipase (MAGL) inhibitor to the subject.

In some embodiments of the method, the MAGL inhibitor does not substantially decrease the analgesic properties of the opioid in the subject. In some embodiments, the MAGL inhibitor delays tolerance to the analgesic properties of the opioid in the subject. In some embodiments, the MAGL inhibitor increases the level of 2-arachidonoylglycerol (2-AG) and/or decreases the level of arachidonic acid (AA) in the subject. In some embodiments, the MAGL inhibitor is co-administered to the subject with the opioid. In some embodiments, the MAGL inhibitor is administered to the subject prior to the administration of the opioid. In some embodiments, the opioid is selected from the group consisting of morphine, codeine, fentanyl, hydrocodone, hydromorphone, meperidine, methadone, and oxycodone.

In some embodiments of the method, the MAGL inhibitor is a small molecule inhibitor. In some embodiments, the MAGL inhibitor is a reversible inhibitor. In some embodiments, the MAGL inhibitor is an irreversible inhibitor. In some embodiments, the MAGL inhibitor is an O-aryl-carbamate and/or benzodioxole compound. In some embodiments, the O-aryl-carbamate and/or benzodioxole compound is JZL-184. In some embodiments, the JZL-184 is administered at a dose of about 10 mg/kg. In some embodiments, the MAGL inhibitor is ABX-1431. In some embodiments, the MAGL inhibitor is a piperazinyl pyrrolidin-2-one compound. In some embodiments, the piperazinyl pyrrolidin-2-one compound is (R)-3t. In some embodiments, the (R)-3t is administered at a dose of about 20 mg/kg. In some embodiments, the MAGL inhibitor decreases the expression, stability, or activity of MAGL. In some embodiments, the MAGL inhibitor decreases the enzymatic activity of MAGL. In some embodiments, the MAGL inhibitor does not substantially inhibit fatty acid amide hydrolase (FAAH). In some embodiments, the MAGL inhibitor reduces or prevents the activation of the nucleus accumbens during morphine-induced conditioned place preference in an animal model.

In some embodiments of the method, the subject is a human. In some embodiments, the subject has an acute or chronic pain condition selected from the group consisting of dental pain, postsurgical pain, musculoskeletal pain, trauma-associated pain, cancer-associated pain, palliative care associated pain, abdominal pain, pelvic pain, infection-associated pain, nephrolithiasis-associated pain, headache, neuropathic pain, arthritis-associated pain, and cholecystitis-associated pain. In some embodiments, the subject has depression, post-traumatic stress disorder, or an anxiety disorder. In some embodiments, the subject is being administered an anti-depressant that acts at least in part through an opioid receptor. In some embodiments, the subject is an adult or an adolescent. In some embodiments, the MAGL inhibitor is administered intravenously, intracranially, intracerebroventricularly, intrathecally, intraspinally, intraperitoneally, intramuscularly, intralesionally, intranasally, orally, or subcutaneously. In some embodiments, the MAGL inhibitor is administered intraperitoneally. In some embodiments, the MAGL inhibitor is administered once or twice per day.

In another aspect, the present disclosure provides a pharmaceutical composition for attenuating the rewarding effect of an opioid in a subject, the composition comprising a therapeutically effective amount of a MAGL inhibitor, an opioid, and a pharmaceutically acceptable carrier.

In some embodiments, the opioid is selected from the group consisting of morphine, codeine, fentanyl, hydrocodone, hydromorphone, meperidine, methadone, tramadol, buprenorphine, and oxycodone. In some embodiments, the MAGL inhibitor is a small molecule inhibitor. In some embodiments, the MAGL inhibitor is a reversible inhibitor. In some embodiments, the MAGL inhibitor is an irreversible inhibitor. In some embodiments, the MAGL inhibitor is an O-aryl-carbamate and/or benzodioxole compound. In some embodiments, the O-aryl-carbamate and/or benzodioxole compound is JZL-184. In some embodiments, the MAGL inhibitor is ABX-1431. In some embodiments, the MAGL inhibitor is a piperazinyl pyrrolidin-2-one compound. In some embodiments, the piperazinyl pyrrolidin-2-one compound is (R)-3t. In some embodiments, the MAGL inhibitor decreases the expression, stability, or activity of MAGL. In some embodiments, the MAGL inhibitor decreases the enzymatic activity of MAGL. In some embodiments, the MAGL inhibitor does not substantially inhibit fatty acid amide hydrolase (FAAH). In some embodiments, the MAGL inhibitor reduces or prevents the activation of the nucleus accumbens during morphine-induced conditioned place preference in an animal model. In some embodiments, the composition is formulated for intravenous, intracranial, intracerebroventricular, intrathecal, intraspinal, intraperitoneal, intramuscular, intralesional, intranasal, oral, or subcutaneous delivery. In some embodiments, the composition is formulated for intraperitoneal delivery.

Other objects, features, and advantages of the present invention will be apparent to one of skill in the art from the following detailed description and figures.

### BRIEF DESCRIPTION OF THE DRAWINGS

FIGS. 1A-1E. Inhibition of MAGL before each morphine conditioning session with JZL-184 abolishes the acquisition of morphine conditioned place preference (CPP). FIG. 1A: Schematic representation of the CPP box used in our studies. FIG. 1B: Experimental timeline of the morphine CPP protocol and pretreatment with JZL-184 before each conditioning session. FIG. 1C: Systemic injection of JZL-184 prior to each morphine conditioning session abolished morphine CPP (Two-way ANOVA, [treatment x day, F(1,26)=19.77, P=0.0001]; Bonferroni post hoc test: Veh: Day 1 vs Day 6 P<0.001***, JZL-184: Day 1 vs Day 6 no significant, Veh n=7, JZL-184 n=8). FIG. 1D: Experimental timeline of the morphine CPP and the JZL-184 injection before the morphine expression test. FIG. 1E: Systemic injection of JZL-184 prior to the test session does not alter the expression of morphine CPP (Two-way ANOVA, [day, F(1,24)=44.82, P=0.0001]; Bonferroni post hoc test: Veh: Day 1 vs Day 6 P<0.001***, JZL-184: Day 1 vs Day 6 P<0.0033**, Veh n=8, JZL-184 n=6).

FIGS. 2A-2D. MAGL inhibition delays morphine tolerance without affecting its analgesic effects. FIG. 2A: Schematic representation of the Tail flick test apparatus used for the antinociception experiments. FIG. 2B: Experimental timeline of the analgesia /tolerance test and the JZL-184 treatment regimen. FIG. 2C: MAGL inhibitor, JZL-184, pretreatment does not affect morphine-induced analgesia. (Two-way ANOVA, [dose; F(4,40)=78.86, P=0.0001, Subjects (matching); F(10,40)=2.316 P=0.0293]; Bonferroni post hoc test: 0.1 mg/kg: Veh vs JZL-184 P>0.999, 0.3 mg/kg: Veh vs JZL-184 P=0.4531, 1 mg/kg: Veh vs JZL-184 P=0.0804, 3.0 mg/kg: Veh vs JZL-184 P>0.999, 10.0 mg/kg: Veh vs JZL-184 P>0.999, Veh n=7, JZL-184 n=5). FIG. 2D: Pretreatment with the MAGL inhibitor, JZL-184, delays the development of tolerance to morphine's analgesic effects (Two-way ANOVA, [treatment x day, F(3,30)=5.007, P=0.0062]; Bonferroni post hoc test: Day1: Veh vs JZL-184 P>0.999, Day3: Veh vs JZL-184 P=0.017^{††}, Day5: Veh vs JZL-184 P=0.0025^{††}, Veh n=7, JZL-184 n=5).

FIGS. 3A-3B. MAGL inhibition before each morphine conditioning session with the reversible MAGL inhibitor (R)-3t inhibits the acquisition of morphine CPP. FIG. 3A: Experimental timeline of morphine CPP and pretreatment with (R)-3t before each conditioning session. FIG. 3B: Systemic injection of (R)-3t abolishes acquisition of CPP (Two-way ANOVA, [day, F(1,40)=15.19, P=0.0004]; Bonferroni post hoc test: Veh: Day 1 vs Day 6 P<0.0057**, (R)-3t: Day 1 vs Day 6 not significant, Veh n=11, (R)-3t n=11).

FIGS. 4A-4G. FAAH inhibition does not affect morphine reward, analgesia, or tolerance. FIGS. 4A, 4 C: Experimental timelines of morphine CPP and pretreatment with the FAAH inhibitor PF-3845 injection before each conditioning session (FIG. 4A) or PF-3845 injection before the morphine CPP expression test (FIG. 4C). FIGS. 4B, 4D: Injection of PF-3845 does not alter acquisition of morphine CPP (FIG. 4B) (Two-way ANOVA, [day, F(1,32)=48.49, P<0.0001]; Bonferroni post hoc test: Veh: Day 1 vs Day 6 P=0.0002***, PF-3845: Day 1 vs Day 6 P=0.0001***, Veh n=9, PF-3845 n=9), nor the expression of morphine CPP (FIG. 4D) (Two-way ANOVA, [day, F(1,34)=26.15, P<0.0001]; Bonferroni post hoc test: Veh: Day 1 vs Day 6 P=0.0004***, PF-3845: Day 1 vs Day 6 P=0.032*, Veh n=11, PF-3845 n=8). FIG. 4E: Experimental timeline of the analgesia/tolerance test and the PF-3845 treatment regimen. FIGS. 4F-4G: Analgesic (FIG. 4F) or morphine tolerance (FIG. 4G) effects are not affected by pretreatment with PF-3845 (FIG. 4F: Two-way ANOVA, [day, F(4,64)=58.62, P<0.0001; Subjects (matching), F(16,64)=2.408 P=0.0068]; Bonferroni post hoc test: All doses: Veh vs PF-3845 P>0.999, Veh n=11, PF-3845 n=7. FIG. 4G: Two-way ANOVA, [day, F(3,48)=16.94, P<0.0001; Subjects (matching), F(16,48)=2.94 P=0.20; Bonferroni post hoc test: All doses: Veh vs PF-3845 not significant Veh n=11, PF-3845 n=7).

FIGS. 5A-5B. Activation of CB1 with the exogenous ligand THC does not alter morphine reward. FIG. 5A: Experimental timeline of the morphine CPP and pretreatment with THC before each conditioning session. FIG. 5B: THC as a pretreatment does not alter the acquisition of morphine CPP (Two-way ANOVA, [day, F(1,28)=61.9, P<0.0001]; Bonferroni post hoc test: Veh: Day 1 vs Day 6 P<0.0001***, THC: Day 1 vs Day 6 P<0.0001***, Veh n=7, THC-compound n=9).

FIGS. 6A-6B. Inhibition of MAGL before each cocaine conditioning session with JZL-184 does not alter cocaine CPP. FIG. 6A: Experimental timeline of the cocaine CPP and pretreatment with JZL-184 before each conditioning session. FIG. 6B: Systemic injection of JZL-184 prior to each cocaine conditioning session does not alter the acquisition of cocaine CPP (Two-way ANOVA, [day, F(1,24)=44.66, P<0.0001]; Bonferroni post hoc test: Veh: Day 1 vs Day 6 P<0.001***, JZL-184: Day 1 vs Day 6 P=0.0052, Veh n=7, JZL-184 n=7).

FIGS. 7A-7D. MAGL inhibition reduces nucleus accumbens (NAc) activity during morphine CPP. FIG. 7A: Experimental timeline for fiber photometry recording during morphinecocaine CPP. FIG. 7B: Brain schematic with an optic fiber implanted into the NAc expressing the calcium indicator GCaMP6s and a representative image of the GCaMP6s expressing in the NAc (aca=anterior commissure, AcbC=nucleus accumbens core, AcbSh=nucleus accumbens shell). FIG. 7C: Graph of the quantification of the mean signal (representative of NAc activity) around zone entry into the morphine- or saline-paired chambers of vehicle- or JZL-184-pretreated animals. (dF/F (%) data for the average signal +/− 5s around zone entries; vehicle-treated animals, morphine vs saline chamber *p<0.05, n=6; JZL-184 treated animals, morphine vs saline p=0.054, n=9). FIG. 7D: Graph of the quantification of the onset of approach to morphine- or saline-paired chambers (dF/F (%) data for the average signal +/− 5s of the onset of approaching a chamber; vehicle-treated animals, morphine vs saline chamber *p<0.05, n=6; JZL-184 treated animals, morphine vs saline p=0.39, n=9).

### DETAILED DESCRIPTION

### 1. Introduction

The present invention is based on the surprising discovery that the inhibition of the monoacylglycerol lipase (MAGL) enzyme can be used to attenuate the rewarding effects of morphine without affecting its analgesic properties. Further, it has been discovered that inhibiting MAGL delays tolerance to the analgesic properties of morphine, again without affecting its analgesic properties. As such, the present disclosure provides methods and compositions that can be used, e.g., for the preparation of adjuvants for use with morphine and other opioid medications when given for analgesic purposes.

### 2. General

Practicing this invention utilizes routine techniques in the field of molecular biology. Basic texts disclosing the general methods of use in this invention include Sambrook and Russell, Molecular Cloning, A Laboratory Manual (3rd ed. 2001); Kriegler, Gene Transfer and Expression: A Laboratory Manual (1990); and Current Protocols in Molecular Biology (Ausubel et al., eds., 1994)).

For nucleic acids, sizes are given in either kilobases (kb), base pairs (bp), or nucleotides (nt). Sizes of single-stranded DNA and/or RNA can be given in nucleotides. These are estimates derived from agarose or acrylamide gel electrophoresis, from sequenced nucleic acids, or from published DNA sequences. For proteins, sizes are given in kilodaltons (kDa) or amino acid residue numbers. Protein sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage and Caruthers, Tetrahedron Lett. 22:1859-1862 (1981), using an automated synthesizer, as described in Van Devanter et al., Nucleic Acids Res. 12:6159-6168 (1984). Purification of oligonucleotides is performed using any art-recognized strategy, e.g., native acrylamide gel electrophoresis or anion-exchange high performance liquid chromatography (HPLC) as described in Pearson and Reanier, J. Chrom. 255: 137-149 (1983).

### 3. Definitions

As used herein, the following terms have the meanings ascribed to them unless specified otherwise.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The terms “about” and “approximately” as used herein shall generally mean an acceptable degree of error for the quantity measured given the nature or precision of the measurements. Typically, exemplary degrees of error are within 20 percent (%), preferably within 10%, and more preferably within 5% of a given value or range of values. Any reference to “about X” specifically indicates at least the values X, 0.8X, 0.81X, 0.82X, 0.83X, 0.84X, 0.85X, 0.86X, 0.87X, 0.88X, 0.89X, 0.9X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, 1.1X, 1.11X, 1.12X, 1.13X, 1.14X, 1.15X, 1.16X, 1.17X, 1.18X, 1.19X, and 1.2X. Thus, “about X” is intended to teach and provide written description support for a claim limitation of, e.g., “0.98X.”

The terms “subject”, “patient”, or “individual” are used herein interchangeably to refer to a human or animal. For example, the animal subject may be a mammal, a primate (e.g., a monkey), a livestock animal (e.g., a horse, a cow, a sheep, a pig, or a goat), a companion animal (e.g., a dog, a cat), a laboratory test animal (e.g., a mouse, a rat, a guinea pig, a bird), an animal of veterinary significance, or an animal of economic significance. In particular embodiments, a “subject”, “patient” or “individual” is a human or animal being treated with an opioid for analgesic purposes, e.g., for the treatment of acute or chronic pain.

“MAGL” (monoacylglycerol lipase, or monoglyceride lipase) is a serine hydrolase that is expressed at high levels in the brain and that catalyzes the conversion of monoacylglycerides such as the endocannabinoid 2-arachidonoylglycerol (2-AG) to free fatty acids, e.g., arachidonic acid, and glycerol. The human enzyme is encoded by the MGLL gene (HGNC ID: 17038, Entrez Gene ID: 11343). The enzyme is a member of the alpha/beta (AB) hydrolase superfamily. Multiple isoforms of the enzyme exist, e.g., in humans, any of which can be targeted using the present methods. For example, any of the human isoforms (e.g., GenBank Accession Nos. XP_024309102.1, XP_024309101.1, XP_016861155.1, XP_016861154.1, XP_016861153.1, XP_016861152.1, XP_016861151.1, XP_011510685.1, XP_011510684.1, XP_011510681.1, XP_011510680.1, XP_011510679.1, NP_001243514.1, ACD37712.1, EAW79331.1, EAW79330.1, EAW79329.1, NP_001003794.1, CAG33116.1, or Q99685.2) can be targeted, as can any isoform with 50%, 60%, 70%, 80%, 85%, 90%, 95%, or higher identity to the amino acid sequences of any of GenBank Accession Nos. XP_024309102.1, XP_024309101.1, XP_016861155.1, XP_016861154.1, XP_016861153.1, XP_016861152.1, XP_016861151.1, XP_011510685.1, XP_011510684.1, XP_011510681.1, XP_011510680.1, XP_011510679.1, NP_001243514.1, ACD37712.1, EAW79331.1, EAW79330.1, EAW79329.1, NP_001003794.1, CAG33116.1, or Q99685.2, or of any other MAGL enzyme from humans or animals.

A “MAGL inhibitor” refers to any agent that is capable of inhibiting, reducing, decreasing, attenuating, abolishing, eliminating, slowing, or counteracting in any way any aspect of the expression, stability, or activity of MAGL. A MAGL inhibitor can, for example, reduce any aspect of the expression, e.g., transcription, RNA processing, RNA stability, or translation of a gene encoding MAGL, e.g., the human MGLL gene, by, e.g., 10%, 15%, 20%, 25%, 30%, 35%, 40%, 45%, 50%, 55%, 60%, 65%, 70%, 75%, 80%, 85%, 90%, 95%, or more as compared to a control, e.g., in the absence of the inhibitor, in vitro or in vivo. Similarly, a MAGL inhibitor can, for example, reduce the activity, e.g., enzymatic activity, of a MAGL enzyme by, e.g., 10%, 15%, 20%, 25%, 30%, 35%, 40%, 45%, 50%, 55%, 60%, 65%, 70%, 75%, 80%, 85%, 90%, 95%, or more as compared to a control, e.g., in the absence of the inhibitor, in vitro or in vivo. Further, a MAGL inhibitor can, for example, reduce the stability of a MAGL enzyme by, e.g., 10%, 15%, 20%, 25%, 30%, 35%, 40%, 45%, 50%, 55%, 60%, 65%, 70%, 75%, 80%, 85%, 90%, 95%, or more as compared to a control, e.g., in the absence of the inhibitor, in vitro or in vivo. An “MAGL inhibitor”, also referred to herein as an “agent” or “compound,” can be any molecule, either naturally occurring or synthetic, e.g., peptide, protein, oligopeptide (e.g., from about 5 to about 25 amino acids in length, e.g., about 5, 10, 15, 20, or 25 amino acids in length), small molecule (e.g., an organic molecule having a molecular weight of less than about 2500 daltons, e.g., less than 2000, less than 1000, or less than 500 daltons), antibody, nanobody, polysaccharide, lipid, fatty acid, inhibitory RNA (e.g., siRNA, shRNA, microRNA), modified RNA, polynucleotide, oligonucleotide, e.g. antisense oligonucleotide, aptamer, affimer, drug compound, or other compound.

“JZL-184” (or “JZL184” or “JZL 184”) is an O-aryl-carbamate and benzodioxole that is an irreversible inhibitor of monoacylglycerol lipase (MAGL). It is highly selective for MAGL over other brain serine hydrolases, including fatty acid amide hydrolase (FAAH). It has the IUPAC name (4-nitrophenyl) 4-[bis(1,3-benzodioxol-5-yl)-hydroxymethyl]piperidine-1-carboxylate, and the structure

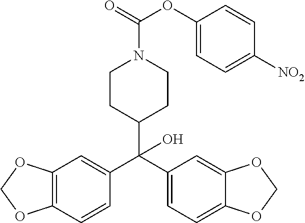 [SMILES: O=C(Oc1ccc([N+](=O)[O-])cc1)N1CCC(C(O)(c2ccc3c(c2)OCO3)c2ccc3c(c2)OCO3)CC1]

The PubChem CID for JZL-184 is 25021165, the entire disclosure of which is herein incorporated by reference.

(R)-3t is a piperazinyl pyrrolidinone that is a reversible MAGL inhibitor described, e.g., in Aida et al. (2018) J. Med. Chem. 61:9205, the entire disclosure of which is herein incorporated by reference. (R)-3t has the structure

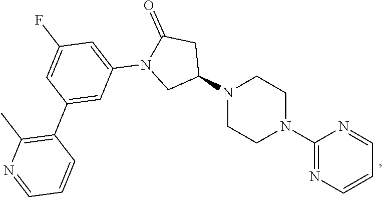 [SMILES: Cc1ncccc1-c1cc(F)cc(N2C[C@H](N3CCN(c4ncccn4)CC3)CC2=O)c1]

and can be synthesized, e.g., as described in Aida et al. (2018).

“2-AG” refers to the endocannabinoid 2-arachidonoylglycerol (PubChem CID: 5282280), which is present at relatively high levels in the central nervous system and which is an endogenous agonist of the cannabinoid receptors CB1 and CB2. 2-AG is hydrolyzed by MAGL to form arachidonic acid (AA) and glycerol. Accordingly, inhibition of MAGL leads to increased levels of 2-AG and, consequently, increased levels of 2-AG signaling, e.g., through the CB1 and CB2 receptors.

“Opioids” are a group of alkaloids that act as analgesic agents and that interact with opioid receptors. As used herein, opioids can include naturally occurring compounds (e.g., morphine, codeine, thebaine, papaverine), semi-synthetic compounds (e.g. diamorphine/heroin, dihydromorphone, buprenorphine, oxycodone) and synthetic compounds (e.g., pethidine, fentanyl, methadone, alfentanil, remifentanil, tapentadol), and can include morphinan derivatives (e.g., levorphanol, butorphanol), diphenylheptane derivatives, (e.g., methadone, propoxyphene), benzomorphan derivatives (e.g., pentazocine, phenazocine), and phenylpiperidine derivatives (pethidine, alfentanil, fentanyl, remifentanil). Opioids act through G-protein coupled opioid receptors, e.g. OP1 (or DOR, or delta), OP2 (or KOR, or kappa) or OP3 (or MOR, or mu), and produce a range of effects, including analgesia. In particular embodiments, an opioid as used herein is selected from the group consisting of morphine, codeine, fentanyl, hydrocodone, hydromorphone, meperidine, methadone, tramadol, buprenorphine, and oxycodone. Opioids also produce rewarding effects, or positive reinforcement related to the administration of the compounds, in particular by acting through the nucleus accumbens and activating the reward system. Opioids can also give rise to tolerance effects, where increasing doses of the compound are required over time to achieve the same level of response (e.g., analgesia) achieved initially. Tolerance to opioids can develop at the neuronal level, where downregulation of adenylate cyclase leads to a decrease in the amount or extent of neuronal firing upon the administration of a given level or dose of the opioid. As used herein, opioids include any natural, semi-synthetic, or synthetic compound that can stimulate one or more opioid receptors in the body and produce analgesia. More broadly, an “opioid” as used herein can be any compound that acts through the opioid receptor to produce a therapeutic effect and that can also lead to a rewarding effect and/or tolerance, including anti-depressants such as ketamine, tianeptine, or buprenorphine/samidorphan.

The terms “expression” and “expressed” refer to the production of a transcriptional and/or translational product, e.g., of a nucleic acid sequence encoding a protein (e.g., MAGL). In some embodiments, the term refers to the production of a transcriptional and/or translational product encoded by a gene (e.g., the human MGLL gene) or a portion thereof. The level of expression of a DNA molecule in a cell may be assessed, e.g., on the basis of either the amount of corresponding mRNA that is present within the cell or the amount of protein encoded by that DNA produced by the cell.

The term “nucleic acid” or “polynucleotide” includes deoxyribonucleotides or ribonucleotides and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Cassol et al. (1992); Rossolini et al., Mol. Cell. Probes 8:91-98 (1994)). The term nucleic acid is used interchangeably with gene, cDNA, and mRNA encoded by a gene.

The terms “polypeptide,” “peptide,” and “protein” are used interchangeably herein to include a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins (i.e., antigens), wherein the amino acid residues are linked by covalent peptide bonds.

The term “amino acid” includes naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs include compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” include chemical compounds that have a structure that is different from the general chemical structure of an amino acid, but that functions in a manner similar to a naturally occurring amino acid.

Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, “conservatively modified variants” include those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions and/or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and/or alleles.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. For example, recombinant cells express genes that are not found within the native (non-recombinant) form of the cell or express native genes that are otherwise abnormally expressed, under-expressed, or not expressed at all.

The term “antibody” refers to a polypeptide that is substantially encoded by an immunoglobulin gene or immunoglobulin genes, or fragments thereof which specifically bind and recognize an analyte (antigen). The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD, and IgE, respectively. An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains, respectively. Variations in amino acid sequences of the variable regions are responsible for the vast diversity of antigen-binding sites, and the greatest variability occurs throughout three hypervariable regions, termed complementary determining regions (CDRs). The tail region of the antibody, known as the Fc region, is comprised of two constant domains (C_{H}2, and C_{H}3) from each of the heavy chains. The Fc region is responsible for recruiting effector functions through binding of F_{C} receptors on neutrophils and macrophages.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially a Fab with part of the hinge region (see, Paul (Ed.) Fundamental Immunology, Third Edition, Raven Press, NY (1993)). While various antibody fragments are defined in terms of the digestion of an intact antibody, one of skill will appreciate that such fragments may be synthesized de novo either chemically or by utilizing recombinant DNA methodology. Thus, the term “antibody,” as used herein, also includes antibody fragments either produced by the modification of whole antibodies or those synthesized de novo using recombinant DNA methodologies (e.g., single chain F_{V}).

The term “humanized antibody” refers to an antibody comprising at least one chain comprising variable region framework residues substantially from a human antibody chain (referred to as the acceptor immunoglobulin or antibody) and at least one complementary determining region (CDR) substantially from a mouse antibody (referred to as the donor immunoglobulin or antibody). See, e.g., Queen et al., Proc. Natl. Acad. Sci. USA 86: 10029 10033 (1989), U.S. Pat. No. 5,530,101, U.S. Pat. No. 5,585,089, U.S. Pat. No. 5,693,761, WO 90/07861, and U.S. Pat. No. 5,225,539. The constant region(s), if present, can also be substantially or entirely from a human immunoglobulin. Methods of making humanized antibodies are known in the art. See, e.g., U.S. Pat. No. 7,256,273.

The phrase “specifically binds,” when used in the context of describing a binding relationship of a particular molecule to a protein or peptide, refers to a binding reaction that is determinative of the presence of the protein in a heterogeneous population of proteins and other biologics. Thus, under designated binding assay conditions, the specified binding agent (e.g., an antibody or small molecule) binds to a particular protein at least two times the background and does not substantially bind in a significant amount to other proteins present in the sample. Specific binding of an antibody or other compound under such conditions may require an antibody (or other compound) that is selected for its specificity for a particular protein or a protein but not its similar “sister” proteins. A variety of immunoassay formats may be used to select antibodies or fragments thereof that are specifically immunoreactive with a particular protein or in a particular form. For example, solid-phase ELISA immunoassays are routinely used to select antibodies that are specifically immunoreactive with a protein (see, e.g., Harlow & Lane, Antibodies, A Laboratory Manual (1988) for a description of immunoassay formats and conditions that can be used to determine specific immunoreactivity). Typically a specific or selective binding reaction will be at least twice background signal or noise and more typically more than 10 to 100 times background.

The term “interfering RNA” or “RNAi” or “interfering RNA sequence” includes single-stranded RNA (e.g., mature miRNA, ssRNAi oligonucleotides, ssDNAi oligonucleotides), double-stranded RNA (i.e., duplex RNA such as small interfering RNA (siRNA), Dicer-substrate dsRNA, shRNA, aiRNA, or pre-miRNA), a DNA-RNA hybrid, or a DNA-DNA hybrid that is capable of reducing or inhibiting the expression of a target gene or sequence (e.g., by mediating the degradation or inhibiting the translation of mRNAs which are complementary to the interfering RNA sequence) when the interfering RNA is in the same cell as the target gene or sequence. Interfering RNA thus refers to the single-stranded RNA that is complementary to a target mRNA sequence or to the double-stranded RNA formed by two complementary strands or by a single, self-complementary strand. Interfering RNA may have substantial or complete identity to the target gene or sequence, or may comprise a region of mismatch (i.e., a mismatch motif). The sequence of the interfering RNA can correspond to the full-length target gene, or a subsequence thereof. In some embodiments, the interfering RNA molecules are chemically synthesized. The interfering RNA molecules may also be generated by cleavage of longer dsRNA (e.g., dsRNA greater than about 25 nucleotides in length) with the E. coli RNase III or Dicer. In other embodiments, the interfering RNA molecules may be encoded by a plasmid (e.g., transcribed as sequences that automatically fold into duplexes with hairpin loops).

The term “treating” or “treatment” refers to any one of the following: ameliorating one or more symptoms of a disease or condition, e.g., reducing pain, attenuating rewarding effects, delaying tolerance; preventing the manifestation of such symptoms before they occur; slowing down or completely preventing the progression of the disease or condition (as may be evident by longer periods between reoccurrence episodes, slowing down or prevention of the deterioration of symptoms, etc.); enhancing the onset of a remission period; slowing down the irreversible damage caused in the progressive-chronic stage of the disease or condition (both in the primary and secondary stages); delaying the onset of said progressive stage; or any combination thereof.

The term “administer,” “administering,” or “administration” refers to the methods that may be used to enable delivery of agents or compositions such as the MAGL inhibitors described herein to a desired site of biological action. These methods include, but are not limited to, parenteral administration (e.g., intravenous, subcutaneous, intraperitoneal, intramuscular, intra-arterial, intravascular, intracardiac, intrathecal, intranasal, intradermal, intravitreal, and the like), intracranial, transmucosal injection, oral administration, administration as a suppository, and topical administration. One skilled in the art will know of additional methods for administering a therapeutically effective amount of the compounds described herein for preventing or relieving one or more symptoms associated with a disease or condition.

The term “therapeutically effective amount” or “therapeutically effective dose” or “effective amount” refers to an amount of a compound (e.g., MAGL inhibitor) that is sufficient to bring about a beneficial or desired clinical effect, e.g., decrease or absence of reward effect of an opioid, delay or prevention of tolerance to an opioid, etc. A therapeutically effective amount or dose may be based on factors individual to each patient, including, but not limited to, the patient's age, size, type or extent of disease or condition, stage of the disease or condition, route of administration, the type or extent of supplemental therapy used, ongoing disease process and type of treatment desired (e.g., aggressive vs. conventional treatment). Therapeutically effective amounts of a pharmaceutical compound or compositions, as described herein, can be estimated initially from cell culture and animal models. For example, ICso values determined in cell culture methods can serve as a starting point in animal models, while ICso values determined in animal models can be used to find a therapeutically effective dose in humans.

The term “pharmaceutically acceptable carrier” refers to refers to a carrier or a diluent that does not cause significant irritation to an organism and does not abrogate the biological activity and properties of the administered compound.

### 4. Detailed Description of the Embodiments

The present disclosure provides methods and compositions for use in conjunction with the administration of an opioid, to attenuate the rewarding effects of the opioid and/or delay tolerance to the analgesic properties of the opioid while maintaining the opioid's analgesic properties. In particular, the methods and compositions involve the inhibition of the MAGL enzyme, which catabolizes, e.g., 2-arachidonoylglycerol (2-AG) to form arachidonic acid and glycerol. Without being bound by the following theory, it is believed that the enhanced 2-AG endocannabinoid signaling resulting from MAGL inhibition acts to attenuate the rewarding effects of opioids and delay tolerance to the analgesic properties of opioids, without substantially affecting the opioids' analgesic properties.

MAGL can be inhibited in any of a number of ways, including by administering a compound, e.g., a small molecule, antibody, RNA molecule, or peptide, that decreases, inhibits, attenuates, abolishes, counteracts, or reduces in any way the activity (e.g., enzymatic activity), stability, or expression of MAGL in a subject. In particular embodiments, MAGL is inhibited by a small molecule that acts in the central nervous system to reduce or eliminate MAGL enzymatic activity. MAGL inhibitors can be used to attenuate the rewarding effects of and/or delay tolerance to the analgesic properties of any opioid, e.g. morphine, codeine, fentanyl, hydrocodone, hydromorphone, meperidine, methadone, tramadol, buprenorphine, or oxycodone, and can be used in subjects being administered an opioid for any reason, including for the treatment of any type of acute or chronic pain or the treatment of depression, post-traumatic stress disorder (PTSD), or an anxiety disorder.

In particular embodiments, the inhibition of MAGL in a subject leads to an increase in the level of 2-arachidonoylglycerol (2-AG) and/or a decrease in arachidonic acid (AA) in the subject, e.g., in the central nervous system of the subject. In particular embodiments, the inhibition of MAGL in a subject leads to increased CB1 or CB2 activity and/or signaling in the subject, e.g., the central nervous system of the subject. In particular embodiments, the MAGL inhibitor is a selective inhibitor that does not, or does not substantially, inhibit the fatty acid amide hydrolase (FAAH) enzyme in the subject. In particular embodiments, the MAGL inhibitor prevents or reduces activation of the nucleus accumbens during morphine-induced conditioned place preference, e.g., as assayed in an animal model such as mice.

In particular embodiments, the herein-described benefits of MAGL inhibition in a subject, e.g., the attenuation of rewarding effects and/or the delay of tolerance to the analgesic properties of an opioid without affecting the analgesic effects of the opioid, occur independently of other potential effects of MAGL inhibition, e.g., reduction of opioid withdrawal, opioid-sparing effects, enhancement of an opioid's antinociceptive effects, etc. In other words, while one or more of these other effects may be present, the herein-described effects on reward and tolerance do not require any other effects and would occur even in their absence. In addition, the herein-described benefits of MAGL inhibition occur due to the elevated levels of 2-AG to strongly engage with the CB1 receptor. Ligands with weaker binding and activation of the CB1 receptor, such as THC, do not attenuate the rewarding effects of an opioid.

### Subjects

The subject can be any subject, e.g. a human or other mammal, that is being or will be administered an opioid for analgesic purposes and/or for the treatment of, e.g., depression, post-traumatic stress disorder (PTSD), or an anxiety disorder. In some embodiments, the subject is a human. In some embodiments, the subject is an adult. In some embodiments, the subject is an adolescent. In some embodiments, the subject is a child. In some embodiments, the subject is female (e.g., an adult or adolescent female). In some embodiments, the subject is male (e.g., an adult or adolescent male).

The subject may or will be receiving opioid administration for any purpose, particularly for analgesic purposes, including for any type or source of acute or chronic pain, including but not limited to dental pain, postsurgical pain, musculoskeletal pain, trauma-associated pain, cancer-associated pain, palliative care associated pain, abdominal pain, pelvic pain, infection-associated pain, nephrolithiasis-associated pain, cholecystitis-associated pain, nociceptive pain, headaches (including migraines), arthritis-associated pain, and neuropathic pain such as diabetic neuropathy. In some embodiments, the opioid is administered to the subject to treat depression, post-traumatic stress disorder (PTSD), or an anxiety disorder. For example, in some embodiments, a MAGL inhibitor is administered to a subject to reduce or attenuate the rewarding effects of an anti-depressant that acts, at least in part, through the opioid system (e.g., through one or more opioid receptors), including, but not limited to, ketamine, tianeptine, or buprenorphine/samidorphan.

The subject may or will be receiving any opioid, including morphine, codeine, fentanyl, hydrocodone, hydromorphone, meperidine, methadone, tramadol, buprenorphine, and oxycodone, through any delivery system (e.g., oral, transdermal, subcutaneous, intravenous, intrathecal, sublingual, buccal, intranasal, rectal, inhaled, topical) at any dose (e.g., 1-100 MME/day, 10-90 MME/day, 5, 10, 15, 20, 25, 30, 35, 40, 45, 50, 55, 60, 65, 70, 75, 80, 85, 90 MME/day), in any form (including rapid onset, extended release, short-acting and long acting forms, tablets, capsules, injectable suspensions, injectable solutions, suppositories, parenteral solutions, transdermal patches), for any duration (e.g., for short-term treatment of acute pain or long-term treatment of chronic pain, e.g., for 1, 2, 3, 4, 5, 6, 7, 8, 9, 10, 20, 30, 40, 50, 60, 70, 80, 90, 100, 150, 200, 250, 300, 350, 365 or more days), and/or at any frequency (e.g., every 4-6 hours, every 8 hours, 1×/day, 2×/day, every 48 hours, every 72 hours, etc.)

In particular embodiments, the subject may display one or more signs indicating that attenuating rewarding effects may be beneficial, e.g., a pattern or history of increasing opioid use or of opioid misuse, and/or one or more signs indicating that the subject is developing or has developed tolerance to the analgesic properties of an opioid, e.g., a pattern of increasing opioid use due to a decrease or loss of analgesic effects of an opioid, in particular where the opioid was previously effective.

### Rewarding Effects

The present methods can be used to attenuate the rewarding effect of an opioid without reducing its analgesic effects. “Attenuate” as used herein can refer to any degree of decrease, inhibition, slowing, or reduction in any feelings of pleasure, any positive associations, or any positive reinforcement associated with the opioid, and/or to any degree of decrease, inhibition, slowing, or reduction in any biological or physiological aspects of reward effects, e.g., activation of the mesolimbic reward system, signaling in the ventral tegmental area of the brain, dopamine release in the nucleus accumbens, activity in the nucleus accumbens as measured, e.g., using fiber photometry with the calcium indicator GCaMP6s, etc. For example, any decrease of any measure of a rewarding effect, e.g., a decrease of 5%, 10%, 15%, 20%, 25%, 30%, 35%, 40%, 45%, 50%, 55%, 60%, 65%, 70%, 75%, 80%, 85%, 90%, 95%, or more, is encompassed by the present methods.

The ability of a MAGL inhibitor to attenuate the rewarding effect of an opioid can also be assessed in animal models. For example, a morphine conditioning place preference (CPP) paradigm can be used as a preclinical model to measure reward, in which mice are conditioned to associate a particular chamber with the rewarding effects of morphine (see, e.g., Example 1). Reward, e.g., as measured by a preference for the morphine chamber, can be assessed at different phases of morphine conditioning, for example to assess if a MAGL inhibitor decreases or inhibits the establishment of the rewarding effect or of its maintenance or expression.

### Delaying Tolerance

The present methods can be used to produce a delay in the development, establishment, or onset of tolerance to the analgesic properties of an opioid in a subject. Tolerance can be assessed using any of a number of methods. For example, an assessment can be made by, e.g., a medical professional, of decreasing effectiveness of the opioid over time as measured or assessed using, e.g., any of the herein-described methods of assessing pain. Such an assessment could indicate, e.g., that a given dose of an opioid is becoming less analgesically effective over time, and/or that an increased dose is required to achieve a previously attained level of analgesic effectiveness. In some embodiments of the present methods, the administration of the MAGL inhibitor results in a delay in the development, establishment, or onset of tolerance, such that the analgesic effectiveness of a given dose of the opioid remains substantially the same over time, or that any decrease that may appear in the analgesic effectiveness of a given dose of the opioid is less severe, has a later onset, or develops more slowly than in the absence of the MAGL inhibitor. Any detectable delay in the onset or development of tolerance, or any detectable decrease in the severity or extent of tolerance, or any delay or decrease of, e.g., 1%, 2%, 3%, 4%, 5%, 6%, 7%, 8%, 9%, 10%, 15%, 20%, 25%, 30%, 40%, 50%, or more in the onset or development of tolerance, in the presence of the MAGL inhibitor is encompassed by the present methods.

### Analgesic Properties

Any method can be used to assess the analgesic efficacy of an opioid, e.g., to confirm that a MAGL inhibitor does not substantially decrease the analgesic properties of the opioid. A non-limiting list of assessment tools that can be used includes: self-reporting by the subject or by a health care professional, family member or caregiver, Visual Analog Scale, Wong-Baker FACES Scale, Indiana Polyclinic Combined Pain Scale, Functional Independence Measure, Alder Hey Triage Pain Score, Behavioral Pain Scale, Brief Pain Inventory, Checklist of Nonverbal Pain Indicators, Clinical Global Impression, Critical Care Pain Observation Tool, COMFORT Scale, Dallas Pain Questionnaire, and the like. Any assessment by any of these or by any equivalent measure, or by a subjective assessment by the subject or a caregiver or medical professional, or by an assessment using any other measure of pain, that the analgesic properties of the opioid are substantially maintained, e.g., have not decreased by more than, e.g., 1%, 2%, 3%, 4%, 5%, 6%, 7%, 8%, 9%, 10%, 11%, 12%, 13%, 14%, 15%, 20%, 25%, or 30%, indicates that the MAGL inhibitor has not substantially decreased the analgesic properties of the opioid.

In some embodiments, the analgesic properties of an opioid are assessed in animals using standard methods. For example, a tail flick test can be used, in which heat is applied to the animal's tail, and the time it takes the animal to flick its tail is recorded. Analgesic effects produce a measurable delay in the response time of the animal, i.e., the time at which it flicks its tail in response to the thermal pain. Another, similar test that can be used is the hot plate test, in which the animal is placed on the heated surface of a plate and the time at which the animal responds to the heat by, e.g., licking its paw or jumping off the plate, is recorded. Similarly, analgesic compounds produce a delay in the response time of the animal to the thermal pain. Any MAGL inhibitor can be used in the present methods that does not result in a substantial decrease, e.g., a decrease of more than, e.g., 1%, 2%, 3%, 4%, 5%, 6%, 7%, 8%, 9%, 10%, 11%, 12%, 13%, 14%, 15%, 20%, 25%, or 30%, in analgesic effectiveness as measured using one of these, or any equivalent, test.

### Assessing MAGL levels

Any of a number of methods can be used to assess the level or activity of MAGL in cells or tissues, e.g., when assessing the efficacy of an inhibitor of MAGL or when assessing the level or activity of MAGL in a subject. For example, the level of MAGL can be assessed by examining the transcription of a gene encoding MAGL (e.g., the MGLL gene), by examining the levels of MAGL protein, by measuring MAGL enzyme activity, or indirectly by measuring, e.g., 2-AG or AA levels or CB1 or CB2 receptor signaling or activity.

In some embodiments, the methods involve the measurement of MAGL enzyme activity, e.g., using standard methods such as incubating a candidate compound in the presence of MAGL and 2-AG in an appropriate reaction buffer and monitoring the generation of AA (see, e.g., Aida et al. (2018) J. Med. Chem. 61:9205-9217; Cisar et al. (2018) J. Med. Chem. 61:9062-9084; the disclosures of which are herein incorporated by reference in their entireties), or by using any of a number of available kits such as the fluorometric Monoacylglycerol Lipase (MAGL) Activity Assay Kit (BioVision).

In some embodiments, the methods involve the detection of MAGL-encoding polynucleotide (e.g., mRNA) expression, which can be analyzed using routine techniques such as RT-PCR, Real-Time RT-PCR, semi-quantitative RT-PCR, quantitative polymerase chain reaction (qPCR), quantitative RT-PCR (qRT-PCR), multiplexed branched DNA (bDNA) assay, microarray hybridization, or sequence analysis (e.g., RNA sequencing (“RNA-Seq”)). Methods of quantifying polynucleotide expression are described, e.g., in Fassbinder-Orth, Integrative and Comparative Biology, 2014, 54:396-406; Thellin et al., Biotechnology Advances, 2009, 27:323-333; and Zheng et al., Clinical Chemistry, 2006, 52:7 (doi: 10/1373/clinchem.2005.065078). In some embodiments, real-time or quantitative PCR or RT-PCR is used to measure the level of a polynucleotide (e.g., mRNA) in a biological sample. See, e.g., Nolan et al., Nat. Protoc, 2006, 1:1559-1582; Wong et al., BioTechniques, 2005, 39:75-75. Quantitative PCR and RT-PCR assays for measuring gene expression are also commercially available (e.g., TaqMan® Gene Expression Assays, ThermoFisher Scientific).

In some embodiments, the methods involve the detection of MAGL protein expression or stability, e.g., using routine techniques such as immunoassays, two-dimensional gel electrophoresis, and quantitative mass spectrometry that are known to those skilled in the art. Protein quantification techniques are generally described in “Strategies for Protein Quantitation,” Principles of Proteomics, 2nd Edition, R. Twyman, ed., Garland Science, 2013. In some embodiments, protein expression or stability is detected by immunoassay, such as but not limited to enzyme immunoassays (EIA) such as enzyme multiplied immunoassay technique (EMIT), enzyme-linked immunosorbent assay (ELISA), IgM antibody capture ELISA (MAC ELISA), and microparticle enzyme immunoassay (MEIA); capillary electrophoresis immunoassays (CEIA); radioimmunoassays (RIA); immunoradiometric assays (IRMA); immunofluorescence (IF); fluorescence polarization immunoassays (FPIA); and chemiluminescence assays (CL). If desired, such immunoassays can be automated. Immunoassays can also be used in conjunction with laser induced fluorescence (see, e.g., Schmalzing et al., Electrophoresis, 18:2184-93 (1997); Bao, J. Chromatogr. B. Biomed. Sci., 699:463-80 (1997)).

### MAGL inhibitors

Any agent that reduces, decreases, counteracts, attenuates, inhibits, blocks, downregulates, or eliminates in any way the expression, stability or activity, e.g., enzymatic activity, of MAGL can be used in the present methods. Inhibitors can be small molecule compounds, peptides, polypeptides, nucleic acids, antibodies, e.g., blocking antibodies or nanobodies, or any other molecule that reduces, decreases, counteracts, attenuates, inhibits, blocks, downregulates, or eliminates in any way the expression, stability and/or activity of MAGL, e.g., the enzymatic activity of MAGL. In particular embodiments, the MAGL inhibitor does not inhibit FAAH (fatty acid amide hydrolase; see, e.g., Gene ID 2166, UniProtKB O00519), e.g. does not substantially (e.g. not by more than 1%, 2%, 3%, 4%, 5%, 6%, 7%, 8%, 9%, 10%, 15%, 20%, 25%, 30%) reduce, decrease, counteract, attenuate, inhibit, block, downregulate, or eliminate in any way the expression, stability, or activity of FAAH, e.g., as assessed using standard methods. The MAGL inhibitors used according to the present methods can be either reversible (e.g., involving non-covalent, reversible binding to MAGL) or irreversible (e.g., involving covalent binding to or otherwise chemically modifying MAGL) inhibitors.

In some embodiments, the MAGL inhibitor decreases the activity, stability or expression of MAGL by at least 10%, 15%, 20%, 25%, 30%, 35%, 40%, 45%, 50%, 55%, 60%, 65%, 70%, 75%, 80%, 85%, 90%, or more relative to a control level, e.g., in the absence of the inhibitor, in vivo or in vitro.

In some embodiments, the MAGL inhibitor is considered effective if the level of expression or stability of a MAGL polypeptide or a MAGL-encoding polynucleotide is decreased by at least 10%, at least 20%, at least 30%, at least 40%, at least 50%, at least 60%, at least 70%, at least 80%, at least 90% or more as compared to the reference value, e.g., the value in the absence of the inhibitor, in vitro or in vivo. In some embodiments, a MAGL inhibitor is considered effective if the level of expression or stability of a MAGL polypeptide or a MAGL-encoding polynucleotide is decreased by at least 1.5-fold, at least 2-fold, at least 3-fold, at least 4-fold, at least 5-fold, at least 6-fold, at least 7-fold, at least 8-fold, at least 9-fold, at least 10-fold or more as compared to the reference value.

For determining whether MAGL protein levels are decreased in the presence of a MAGL inhibitor, the method comprises comparing the level of the protein (e.g., MAGL protein) in the presence of the inhibitor to a reference value, e.g., the level in the absence of the inhibitor. In some embodiments, a MAGL protein is decreased in the presence of an inhibitor if the level of the MAGL protein is decreased by at least 10%, at least 20%, at least 30%, at least 40%, at least 50%, at least 60%, at least 70%, at least 80%, at least 90% or more as compared to the reference value. In some embodiments, a MAGL protein is decreased in a the presence of an inhibitor if the level of the MAGL protein is decreased by at least 1.5-fold, at least 2-fold, at least 3-fold, at least 4-fold, at least 5-fold, at least 6-fold, at least 7-fold, at least 8-fold, at least 9-fold, at least 10-fold or more as compared to the reference value.

In some embodiments, the MAGL inhibitor is considered effective if the level of MAGL enzymatic activity is decreased by at least 10%, at least 20%, at least 30%, at least 40%, at least 50%, at least 60%, at least 70%, at least 80%, at least 90% or more as compared to the reference value, e.g., the value in the absence of the inhibitor, in vitro or in vivo. In some embodiments, a MAGL inhibitor is considered effective if the level of MAGL enzymatic activity is decreased by at least 1.5-fold, at least 2-fold, at least 3-fold, at least 4-fold, at least 5-fold, at least 6-fold, at least 7-fold, at least 8-fold, at least 9-fold, at least 10-fold or more as compared to the reference value.

In some embodiments, the MAGL inhibitor is capable of crossing the blood-brain barrier (BBB), e.g., so as to act on MAGL in the central nervous system. In some embodiments, e.g., with certain small molecule inhibitors, the inhibitor can naturally cross the BBB, and in some embodiments, e.g., for methods involving the development, selection, and/or use of a MAGL inhibitor, a step may be added in which a candidate inhibitor is evaluated regarding its ability to cross the BBB, e.g., using microfluidic devices comprising BBB models (see, e.g., Wevers et al. (2018) Fluids Barriers CNS 15(1):23), wherein an inhibitor is selected based on its ability to cross the BBB. In some embodiments, the inhibitor may be modified, derivatized, or coupled with an appropriate carrier so as to enable its passage across the BBB. For example, molecules can be chemically modified to form a prodrug, they can be coupled with mannitol or an aromatic substance, an appropriate chemical drug delivery system or carrier with the ability to cross the BBB can be used, they can be linked to an appropriate ligand and transported across the BBB using receptor-mediated mechanisms, they can be transported across the BBB using nanoparticles or liposomes, etc. Any of these approaches, or any other known method for enhancing drug delivery to the CNS, can be used in the present methods. See, e.g., He et al. (2018) Cells 7(4):24; Fu (2018) Adv. Exp. Med. Biol. (2018) 1097:235-259; Azad et al. (2015) Neurosurg Focus 38(3):E9; Wang et al. (2019) Drug Deliv. 26(1):551-565; Patel et al. (2017) CNS Drugs 31(2):109-133; Li et al. (J. Drug Target 25(1):17-28, the disclosures of which are herein incorporated by reference in their entireties.

### Small Molecules

In particular embodiments of the invention, MAGL is inhibited by the administration of a small molecule inhibitor. Any small molecule inhibitor can be used that reduces, e.g., by 10%, 15%, 20%, 25%, 30%, 35%, 40%, 45%, 50%, 55%, 60%, 65%, 70%, 75%, 80%, 85%, 90%, or more, the expression, stability or activity of MAGL relative to a control, e.g., the expression, stability or activity in the absence of the inhibitor. Both irreversible and reversible inhibitors of MAGL can be used. In particular embodiments, a small molecule inhibitor is used that can reduce the enzymatic activity of MAGL in vitro or in vivo. In particular embodiments, the MAGL small molecule inhibitor does not, or does not significantly or substantially, inhibit other serine hydrolases, e.g., FAAH (Fatty Acid Amide Hydrolase).

In particular embodiments, a small molecule inhibitor is used and is formulated together with an opioid, e.g., in a single pharmaceutical composition comprising an opioid, a MAGL inhibitor, and a pharmaceutically acceptable carrier. It will be appreciated, however, that small molecule inhibitors can also be administered independently of the opioid, e.g., in a separate formulation that is administered according to an independent regimen.

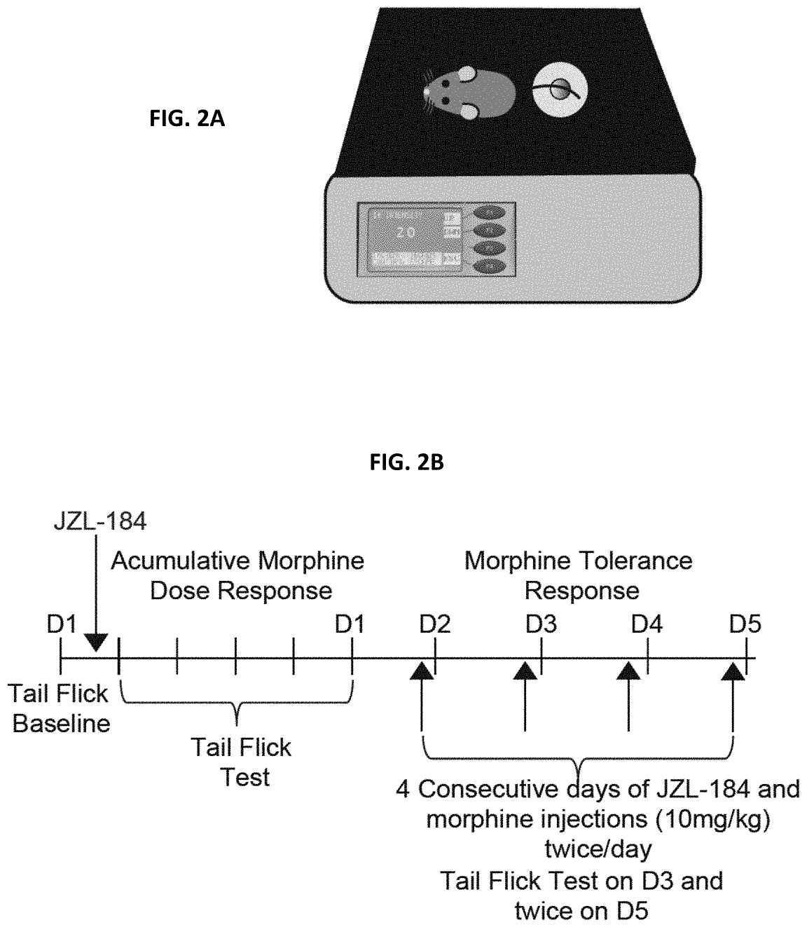

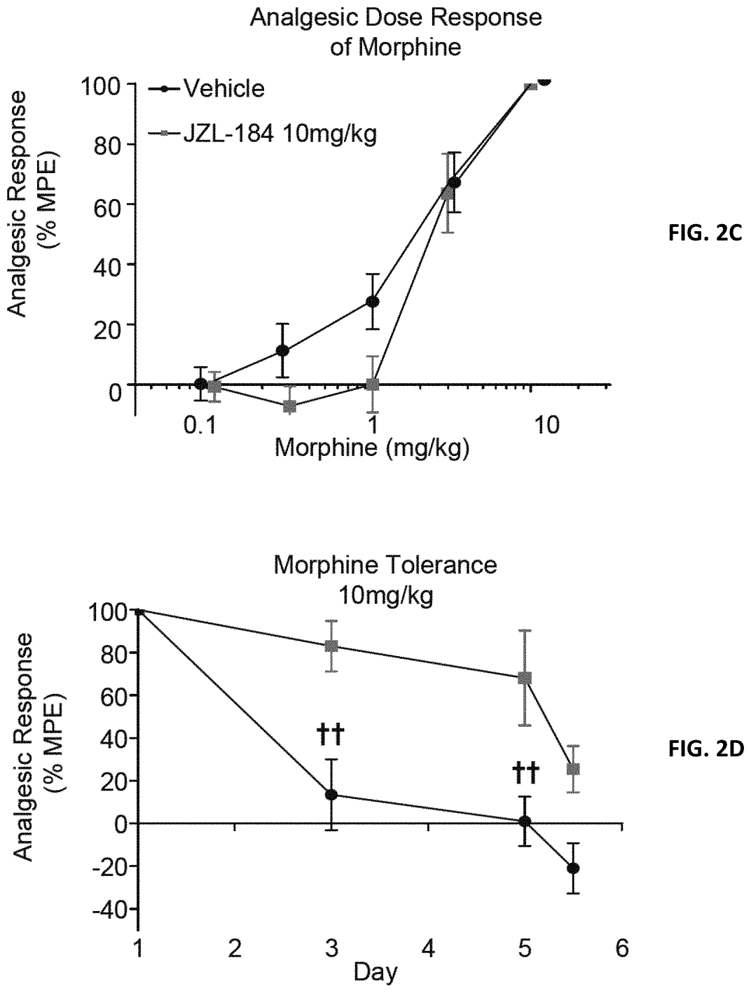

In some embodiments, the small molecule inhibitor is an O-aryl carbamate and/or benzodioxole compound such as JZL-184 (4-nitrophenyl-4-[bis(1,3-benzodioxol-5-yl)(hydroxy)methyl]piperidine-1-carboxylate; PubChem CID 25021165), or any of the compounds disclosed in Long et al. (2009) Nat. Chem. Biol. 5:37-44, the disclosure of which is herein incorporated by reference in its entirety. JZL-184 can be prepared, e.g., as described in Long et al. (2009), or obtained from commercial sources (e.g., Sigma Aldrich, Cayman Chemical, Enzo Life Sciences, and others). In some embodiments, the small molecule inhibitor is a hexafluoroisopropyl carbamate or an O-hexafluoroisopropyl (HFIP) carbamate compound such as ABX-1431 (1,1,1,3,3,3 -hexafluoropropan-2-yl 4-[[2-pyrrolidin-1-yl-4-(trifluoromethyl)-phenyl]methyl]-piperazine-1- carboxylate; PubChem CID 71657619; CAS No. 1446817-84-0), ABD-1970 (1,1,1,3,3,3 -hexafluoropropan-2-yl 4-[[4-chloro-2-(8-oxa-3-azabicyclo[3.2.1]octan-3-yl)phenyl]methyl]piperazine-1-carboxylate; CAS No. 2010154-82-0), JW 642 (4[(3-Phenoxyphenyl)methyl]-1-piperazinecarboxylic acid 2,2,2-trifluoro-1-(trifluoromethyl)ethyl ester; PubChem CID 71656520), CPD-4645 (see, e.g., FIG. 2a of Piro et al. (2018) Journal Neuroinflamm. 15: 142), or any of the compounds disclosed in Cisar et al. (2018) J. Med. Chem 61(20):9062-9084, Chang et al. (2012), Chem. Biol. 19(5):579-588, Piro et al. (2018) Journal Neuroinflamm. 15: 142, or Terrone et al. (2018) Epilepsia 59(1): 79-91, the disclosures of which are herein incorporated by reference in their entireties.

In some embodiments, the small molecule inhibitor is an O-N-hydroxysuccinimidyl (NHS) carbamate compound such as MJN110 (2,5-dioxopyrrolidin-1-yl 4-(bis(4-chlorophenyl)methyl)piperazine-1-carboxylate; PubChem CID 71722059) or any of the compounds disclosed in Chang et al. 2013 ACS Chem Biol. 8(7):1590-1599 or Niphakis et al. 2013 ACS Chem Neurosci. 4(9):1322-32, the disclosures of which are herein incorporated by reference in their entireties.

In some embodiments, the small molecule inhibitor is a maleimide, urea, ketone, carbamate, oxadiazolone, amide derivative, pyrrolidinone derivative, pyrrolo-pyrrole carbamate, 4-(piperazin-1-yl)-pyrrolidin-2-one, or piperazinyl pyrrolidin-2-one compound, such as (R)-3t [(2-Pyrrolidinone, 1-[3-fluoro-5-(2-methyl-3-pyridinyl)phenyl]-4-[4-(2-pyrimidinyl)-1-piperazinyl]-(4R)-)], (4R)-1-[3-fluoro-5-(2-methylpyridin-3-yl)phenyl]-4-[4-(pyrimidin-2-yl)piperazin-1-yl]pyrrolidin-2-one, or any of the compounds disclosed in Aida et al. (2018) J. Med. Chem. 61:9205-9217, WO2015003002, WO2015099196, or WO2017170830, the entire disclosures of which are herein incorporated by reference. (R)-3t can be synthesized, e.g., as described in Aida et al. (2018) or can be obtained from commercial suppliers (e.g., Enamine). In some embodiments, the small molecule inhibitor is a triazolopyridine carboxamide derivative, a triazolopyrimidine carboxamide derivative, or any of the compounds disclosed in WO2008145843, the disclosure of which is herein incorporated by reference in its entirety.

In some embodiments, the small molecule inhibitor is a 1,3-benzoxazol-2(3H)-one compound or any of the compounds disclosed in WO2013174508, the disclosure of which is herein incorporated by reference in its entirety. In some embodiments, the small molecule inhibitor is an E,E-diene compound, or any of the compounds disclosed in WO2013049332 or WO2012069605, the disclosures of which are herein incorporated by reference in their entireties. In some embodiments, the small molecule inhibitor is any of the compounds disclosed in WO2013049293 or Granchi et al. 2017 Expert Opin Ther Pat 27(12):1341-1351, the disclosures of which are herein incorporated by reference in their entireties. In some embodiments, the small molecule inhibitor is PF-06818883, ABX-1626, ABX-1762, or ABX-1772. In some embodiments, the small molecule inhibitor is a combination, derivative, isomer, or tautomer of any one or more of the herein-disclosed compounds.

Suitable small molecule inhibitors (or other inhibitors described herein), e.g., those molecules described herein and derivatives, analogs, variants, and isomers thereof, can be identified by one of skill in the art, e.g., using the herein-described assays for MAGL inhibitors such as assays to measure or detect: a decrease in MAGL expression, MAGL protein stability, or MAGL enzyme activity, an absence of effects on FAAH enzyme activity, a decrease in reward effect when administered with an opioid, maintenance of analgesia when administered with an opioid, prevention or reduction of the development of tolerance when administered with an opioid, prevention or reduction of nucleus accumbens activation in morphine-induced CPP, ability to cross the blood-brain barrier, etc. In some embodiments, an inhibitor provides an improvement in one or more of these aspects (e.g., a decrease in MAGL expression or enzyme activity, decrease in reward effect, etc.) of 10%, 20%, 30%, 40%, 50%, or more, relative to the level in the absence of the inhibitor.

### Inhibitory Nucleic Acids

In some embodiments, the agent comprises an inhibitory nucleic acid, e.g., antisense DNA or RNA, small interfering RNA (siRNA), microRNA (miRNA), or short hairpin RNA (shRNA). In some embodiments, the inhibitory RNA targets a sequence that is identical or substantially identical (e.g., at least 70%, at least 75%, at least 80%, at least 85%, at least 90%, at least 91%, at least 92%, at least 93%, at least 94%, at least 95%, at least 96%, at least 97%, at least 98%, or at least 99% identical) to a target sequence in a MAGL-encoding polynucleotide (e.g., a portion comprising at least 20, at least 30, at least 40, at least 50, at least 60, at least 70, at least 80, at least 90, or at least 100 contiguous nucleotides, e.g., from 20-500, 20-250, 20-100, 50-500, or 50-250 contiguous nucleotides of a MAGL-encoding polynucleotide sequence (e.g., the human MGLL gene, Gene ID: 11343, including of any of its transcript variants, e.g., as set forth in GenBank Accession Nos. BC000551.2, NM_001003794.2, NM_001256585.1, or NM_007283.6).

In some embodiments, the methods described herein comprise treating a subject, e.g., a subject being administered an opioid for analgesic purposes, using an shRNA or siRNA. A shRNA is an artificial RNA molecule with a hairpin turn that can be used to silence target gene expression via the siRNA it produces in cells. See, e.g., Fire et al., Nature 391:806-811, 1998; Elbashir et al., Nature 411:494-498, 2001; Chakraborty et al., Mol Ther Nucleic Acids 8:132-143, 2017; and Bouard et al., Br. J. Pharmacol. 157:153-165, 2009. In some embodiments, a method of treating a subject being administered an opioid for analgesic purposes comprises administering to the subject a therapeutically effective amount of a modified RNA or a vector comprising a polynucleotide that encodes an shRNA or siRNA capable of hybridizing to a portion of a MAGL mRNA (e.g., a portion of the huma MAGL-encoding polynucleotide sequence set forth in any of GenBank Accession Nos. BC000551.2, NM_001003794.2, NM_001256585.1, or NM_007283.6). In some embodiments, the vector further comprises appropriate expression control elements known in the art, including, e.g., promoters (e.g., inducible promoters or tissue specific promoters), enhancers, and/or transcription terminators.

In some embodiments, the agent is a MAGL-specific microRNA (miRNA or miR). A microRNA is a small non-coding RNA molecule that functions in RNA silencing and post-transcriptional regulation of gene expression. miRNAs base pair with complementary sequences within the mRNA transcript. As a result, the mRNA transcript may be silenced by one or more of the mechanisms such as cleavage of the mRNA strand, destabilization of the mRNA through shortening of its poly(A) tail, and decrease in the translation efficiency of the mRNA transcript into proteins by ribosomes.

In some embodiments, the agent is an antisense oligonucleotide, e.g., an RNase H-dependent antisense oligonucleotide (ASO). ASOs are single-stranded, chemically modified oligonucleotides that bind to complementary sequences in target mRNAs and reduce gene expression both by RNase H-mediated cleavage of the target RNA and by inhibition of translation by steric blockade of ribosomes. In some embodiments, the oligonucleotide is capable of hybridizing to a portion of a MAGL mRNA (e.g., a portion of a huma MAGL-encoding polynucleotide sequence as set forth in any of GenBank Accession Nos. BC000551.2, NM_001003794.2, NM_001256585.1, or NM_007283.6). In some embodiments, the oligonucleotide has a length of about 10-30 nucleotides (e.g., 10, 12, 14, 16, 18, 20, 22, 24, 26, 28, or 30 nucleotides). In some embodiments, the oligonucleotide has 100% complementarity to the portion of the mRNA transcript it binds. In other embodiments, the DNA oligonucleotide has less than 100% complementarity (e.g., 95%, 90%, 85%, 80%, 75%, or 70% complementarity) to the portion of the mRNA transcript it binds, but can still form a stable RNA:DNA duplex for the RNase H to cleave the mRNA transcript.

Suitable antisense molecules, siRNA, miRNA, and shRNA can be produced by standard methods of oligonucleotide synthesis or by ordering such molecules from a contract research organization or supplier by providing the polynucleotide sequence being targeted. The manufacture and deployment of such antisense molecules in general terms may be accomplished using standard techniques described in contemporary reference texts: for example, Gene and Cell Therapy: Therapeutic Mechanisms and Strategies, 4^{th} edition by N. S. Templeton; Translating Gene Therapy to the Clinic: Techniques and Approaches, 1^{st} edition by J. Laurence and M. Franklin; High-Throughput RNAi Screening: Methods and Protocols (Methods in Molecular Biology) by D. O. Azorsa and S. Arora; and Oligonucleotide-Based Drugs and Therapeutics: Preclinical and Clinical Considerations by N. Ferrari and R. Segui.

Inhibitory nucleic acids can also include RNA aptamers, which are short, synthetic oligonucleotide sequences that bind to proteins (see, e.g., Li et al., Nuc. Acids Res. (2006), 34:6416-24). They are notable for both high affinity and specificity for the targeted molecule, and have the additional advantage of being smaller than antibodies (usually less than 6 kD). RNA aptamers with a desired specificity are generally selected from a combinatorial library, and can be modified to reduce vulnerability to ribonucleases, using methods known in the art.

### Antibodies

In some embodiments, the agent is an anti-MAGL antibody or an antigen-binding fragment thereof. In some embodiments, the antibody is a blocking antibody (i.e., an antibody that binds to a target and directly interferes with the target's function, e.g., MAGL enzyme activity). In some embodiments, the antibody is a neutralizing antibody (i.e., an antibody that binds to a target and negates the downstream cellular effects of the target). In some embodiments, the antibody binds to huma MAGL. In some embodiments, the antibody has been engineered to enhance its ability to cross the blood brain barrier (see, e.g., Neves et al. (2016) Trends in Biotechnology 34(1):36-48, the disclosure of which is herein incorporated by reference in its entirety).

In some embodiments, the antibody is a monoclonal antibody. In some embodiments, the antibody is a polyclonal antibody. In some embodiments, the antibody is a chimeric antibody. In some embodiments, the antibody is a humanized antibody. In some embodiments, the antibody is a human antibody. In some embodiments, the antibody is an antigen-binding fragment, such as a F(ab′)2, Fab′, Fab, scFv, and the like. The term “antibody or antigen-binding fragment” can also encompass multi-specific and hybrid antibodies, with dual or multiple antigen or epitope specificities.

In some embodiments, an anti-MAGL antibody comprises a heavy chain sequence or a portion thereof, and/or a light chain sequence or a portion thereof, of an antibody sequence disclosed herein. In some embodiments, an anti-MAGL antibody comprises one or more complementarity determining regions (CDRs) of an anti-MAGL antibody as disclosed herein. In some embodiments, an anti-MAGL antibody is a nanobody, or single-domain antibody (sdAb), comprising a single monomeric variable antibody domain, e.g., a single VHH domain.

For preparing an antibody that binds to MAGL, many techniques known in the art can be used. See, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2nd ed. 1986)). In some embodiments, antibodies are prepared by immunizing an animal or animals (such as mice, rabbits, or rats) with an antigen for the induction of an antibody response. In some embodiments, the antigen is administered in conjugation with an adjuvant (e.g., Freund's adjuvant). In some embodiments, after the initial immunization, one or more subsequent booster injections of the antigen can be administered to improve antibody production. Following immunization, antigen-specific B cells are harvested, e.g., from the spleen and/or lymphoid tissue. For generating monoclonal antibodies, the B cells are fused with myeloma cells, which are subsequently screened for antigen specificity.

The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Additionally, phage or yeast display technology can be used to identify antibodies and heteromeric Fab fragments that specifically bind to selected antigens (see, e.g., McCafferty et al., Nature 348:552-554 (1990); Marks et al., Biotechnology 10:779-783 (1992); Lou et al.m PEDS 23:311 (2010); and Chao et al., Nature Protocols, 1:755-768 (2006)). Alternatively, antibodies and antibody sequences may be isolated and/or identified using a yeast-based antibody presentation system, such as that disclosed in, e.g., Xu et al., Protein Eng Des Sel, 2013, 26:663-670; WO 2009/036379; WO 2010/105256; and WO 2012/009568. Random combinations of the heavy and light chain gene products generate a large pool of antibodies with different antigenic specificity (see, e.g., Kuby, Immunology (3rd ed. 1997)). Techniques for the production of single chain antibodies or recombinant antibodies (U.S. Pat. No. 4,946,778, U.S. Pat. No. 4,816,567) can also be adapted to produce antibodies.

Antibodies can be produced using any number of expression systems, including prokaryotic and eukaryotic expression systems. In some embodiments, the expression system is a mammalian cell, such as a hybridoma, or a CHO cell. Many such systems are widely available from commercial suppliers. In embodiments in which an antibody comprises both a VH and VL region, the VH and VL regions may be expressed using a single vector, e.g., in a di-cistronic expression unit, or be under the control of different promoters. In other embodiments, the VH and VL region may be expressed using separate vectors.

In some embodiments, an anti-MAGL antibody comprises one or more CDR, heavy chain, and/or light chain sequences that are affinity matured. For chimeric antibodies, methods of making chimeric antibodies are known in the art. For example, chimeric antibodies can be made in which the antigen binding region (heavy chain variable region and light chain variable region) from one species, such as a mouse, is fused to the effector region (constant domain) of another species, such as a human. As another example, “class switched” chimeric antibodies can be made in which the effector region of an antibody is substituted with an effector region of a different immunoglobulin class or subclass.

In some embodiments, an anti-MAGL antibody comprises one or more CDR, heavy chain, and/or light chain sequences that are humanized. For humanized antibodies, methods of making humanized antibodies are known in the art. See, e.g., U.S. Pat. No. 8,095,890. Generally, a humanized antibody has one or more amino acid residues introduced into it from a source which is non-human. As an alternative to humanization, human antibodies can be generated. As a non-limiting example, transgenic animals (e.g., mice) can be produced that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggermann et al., Year in Immun., 7:33 (1993); and U.S. Pat. Nos. 5,591,669, 5,589,369, and 5,545,807.

In some embodiments, antibody fragments (such as a Fab, a Fab′, a F(ab′)2, a scFv, nanobody, or a diabody) are generated. Various techniques have been developed for the production of antibody fragments, such as proteolytic digestion of intact antibodies (see, e.g., Morimoto et al., J. Biochem. Biophys. Meth., 24:107-117 (1992); and Brennan et al., Science, 229:81 (1985)) and the use of recombinant host cells to produce the fragments. For example, antibody fragments can be isolated from antibody phage libraries. Alternatively, Fab′-SH fragments can be directly recovered from E. coli cells and chemically coupled to form F(ab′)2 fragments (see, e.g., Carter et al., BioTechnology, 10:163-167 (1992)). According to another approach, F(ab′)2 fragments can be isolated directly from recombinant host cell culture. Other techniques for the production of antibody fragments will be apparent to those skilled in the art.

Methods for measuring binding affinity and binding kinetics are known in the art. These methods include, but are not limited to, solid-phase binding assays (e.g., ELISA assay), immunoprecipitation, surface plasmon resonance (e.g., Biacore™ (GE Healthcare, Piscataway, N.J.)), kinetic exclusion assays (e.g., KinExA®), flow cytometry, fluorescence-activated cell sorting (FACS), BioLayer interferometry (e.g., Octet™ (FortéBio, Inc., Menlo Park, Calif.)), and western blot analysis.

### Peptides

In some embodiments, the agent is a peptide, e.g,. a peptide that binds to and/or inhibits the enzymatic activity or stability of MAGL. In some embodiments, the agent is a peptide aptamer. Peptide aptamers are artificial proteins that are selected or engineered to bind to specific target molecules. Typically, the peptides include one or more peptide loops of variable sequence displayed by the protein scaffold. Peptide aptamer selection can be made using different systems, including the yeast two-hybrid system. Peptide aptamers can also be selected from combinatorial peptide libraries constructed by phage display and other surface display technologies such as mRNA display, ribosome display, bacterial display and yeast display. See, e.g., Reverdatto et al., 2015, Curr. Top. Med. Chem. 15:1082-1101.

In some embodiments, the agent is an affimer. Affimers are small, highly stable proteins, typically having a molecular weight of about 12-14 kDa, that bind their target molecules with specificity and affinity similar to that of antibodies. Generally, an affimer displays two peptide loops and an N-terminal sequence that can be randomized to bind different target proteins with high affinity and specificity in a similar manner to monoclonal antibodies. Stabilization of the two peptide loops by the protein scaffold constrains the possible conformations that the peptides can take, which increases the binding affinity and specificity compared to libraries of free peptides. Affimers and methods of making affimers are described in the art. See, e.g., Tiede et al., eLife, 2017, 6:e24903. Affimers are also commercially available, e.g., from Avacta Life Sciences.

### Vectors and modified RNA

In some embodiments, polynucleotides providing MAGL inhibiting activity, e.g., a nucleic acid inhibitor such as an siRNA or shRNA, or a polynucleotide encoding a polypeptide that inhibits MAGL, are introduced into cells of a subject using an appropriate vector. Examples of delivery vectors that may be used with the present disclosure are viral vectors, plasmids, exosomes, liposomes, bacterial vectors, biomaterial vectors, or nanoparticles. In some embodiments, any of the herein-described MAGL inhibitors, e.g., a nucleic acid inhibitor or a polynucleotide encoding a polypeptide inhibitor, are introduced into cells using vectors such as viral vectors. Suitable viral vectors include but not limited to adeno-associated viruses (AAVs), adenoviruses, and lentiviruses. In some embodiments, a MAGL inhibitor, e.g., a nucleic acid inhibitor or a polynucleotide encoding a polypeptide inhibitor, is provided in the form of an expression cassette, typically recombinantly produced, having a promoter operably linked to the polynucleotide sequence encoding the inhibitor. In some cases, the promoter is a universal promoter that directs gene expression in all or most tissue types; in other cases, the promoter is one that directs gene expression specifically in cells of the tissue being targeted.

In some embodiments, the nucleic acid inhibitors or nucleic acids encoding protein inhibitors of MAGL are introduced into a subject using modified RNA. Various modifications of RNA are known in the art to enhance, e.g., the translation, potency and/or stability of RNA, e.g., shRNA or mRNA encoding a MAGL polypeptide inhibitor, when introduced into cells of a subject. In particular embodiments, modified mRNA (mmRNA) is used, e.g., mmRNA encoding a polypeptide inhibitor of MAGL. In other embodiments, modified RNA comprising an RNA inhibitor of MAGL expression is used, e.g., siRNA, shRNA, or miRNA. Non-limiting examples of RNA modifications that can be used include anti-reverse-cap analogs (ARCA), polyA tails of, e.g., 100-250 nucleotides in length, replacement of AU-rich sequences in the 3′UTR with sequences from known stable mRNAs, and the inclusion of modified nucleosides and structures such as pseudouridine, e.g., N1-methylpseudouridine, 2-thiouridine, 4′thioRNA, 5-methylcytidine, 6-methyladenosine, amide 3 linkages, thioate linkages, inosine, 2′-deoxyribonucleotides, 5-Bromo-uridine and 2′-O-methylated nucleosides. A non-limiting list of chemical modifications that can be used can be found, e.g., in the online database crdd.osdd.net/servers/sirnamod/. RNAs can be introduced into cells in vivo using any known method, including, inter alia, physical disturbance, the generation of RNA endocytosis by cationic carriers, electroporation, gene guns, ultrasound, nanoparticles, conjugates, or high-pressure injection. Modified RNA can also be introduced by direct injection, e.g., in citrate-buffered saline. RNA can also be delivered using self-assembled lipoplexes or polyplexes that are spontaneously generated by charge-to-charge interactions between negatively charged RNA and cationic lipids or polymers, such as lipoplexes, polyplexes, polycations and dendrimers. Polymers such as poly-L-lysine, polyamidoamine, and polyethyleneimine, chitosan, and poly(β-amino esters) can also be used. See, e.g., Youn et al. (2015) Expert Opin Biol Ther, Sep 2; 15(9): 1337-1348; Kaczmarek et al. (2017) Genome Medicine 9:60; Gan et al. (2019) Nature comm. 10: 871; Chien et al. (2015) Cold Spring Harb Perspect Med. 2015;5:a014035; the entire disclosures of each of which are herein incorporated by reference.

### Administration

The compounds of the present invention can be administered locally in the subject or systemically. In some embodiments, the compounds can be administered, for example, intraperitoneally, intramuscularly, intra-arterially, orally, intravenously, intracranially, intrathecally, intraspinally, intralesionally, intranasally, subcutaneously, intracerebroventricularly, topically, and/or by inhalation.

In some embodiments, the compound is administered in accordance with an acute regimen. In certain instances, the compound is administered to the subject once. In other instances, the compound is administered at one time point, and administered again at a second time point. It will be appreciated that an administration event as described herein, e.g., when the compound is said to be administered once per day, twice per day, at one time point, etc., can comprise a brief or immediate administration event such as an injection or oral dose, or a longer administration procedure such as an IV infusion over several hours. In some instances, the compound is administered to the subject repeatedly (e.g., once or twice daily) as intermittent doses over a short period of time (e.g., 2 days, 3 days, 4 days, 5 days, 6 days, a week, 2 weeks, 3 weeks, 4 weeks, a month, or more). In some cases, the time between compound administrations is about 1 day, 2 days, 3 days, 4 days, 5 days, 6 days, a week, 2 weeks, 3 weeks, 4 weeks, a month, or more. In particular embodiments, the MAGL inhibitor is administered once or twice per day. In some embodiments, the compound is administered continuously or chronically in accordance with a chronic regimen over a desired period of time, e.g., for the same period of time that the opioid is administered. For instance, the compound can be administered such that the amount or level of the compound is substantially constant over a selected time period, e.g. the time period corresponding to the period during which the opioid is administered.

In particular embodiments, the compound is administered together with an opioid, e.g., is co-formulated with the opioid in a single pharmaceutical composition. In some embodiments, the compound is administered independently, e.g., as a separate pharmaceutical composition, either at the same time as the administration of the opioid or according to an independent treatment regimen, to a patient that receiving opioid treatment for analgesic purposes. For example, the compound could be administered before the opioid or at the same time as the opioid. It will be appreciated that any MAGL inhibitor can be administered with any opioid including, in particular embodiments, an opioid selected from the group consisting of morphine, codeine, fentanyl, hydrocodone, hydromorphone, meperidine, methadone, tramadol, buprenorphine, and oxycodone.

Administration of the compound to a subject can be accomplished by methods generally used in the art. The quantity of the compound introduced will take into consideration factors such as sex, age, weight, the types of disease or disorder, stage of the disorder, and the quantity needed to produce the desired result. Generally, for administering the compound for therapeutic purposes, the compound is given at a pharmacologically effective dose. By “pharmacologically effective amount” or “pharmacologically effective dose” is an amount sufficient to produce the desired physiological effect or amount capable of achieving the desired result, particularly for treating the condition or disease, including reducing or eliminating one or more symptoms or manifestations of the condition or disease.

### Pharmaceutical Compositions

The present disclosure provides compositions comprising the MAGL inhibitors as described herein. In some embodiments, the present disclosure provides compositions comprising a MAGL inhibitor as described herein and an opioid. In some embodiments, compositions are provided comprising an opioid and a small molecule inhibitor of MAGL. In some such embodiments, the opioid is morphine. In some such embodiments, the small molecule inhibitor is JZL-184 or (R)-3t.

The pharmaceutical compositions of the compounds of the present invention may comprise a pharmaceutically acceptable carrier. For example, in some embodiments, the compositions comprise a MAGL inhibitor, an opioid, and a pharmaceutically acceptable carrier. In certain aspects, pharmaceutically acceptable carriers are determined in part by the particular composition being administered, as well as by the particular method used to administer the composition. Accordingly, there is a wide variety of suitable formulations of pharmaceutical compositions of the present invention (see, e.g., REMINGTON'S PHARMACEUTICAL SCIENCES, 18TH ED., Mack Publishing Co., Easton, Pa. (1990)).

As used herein, “pharmaceutically acceptable carrier” comprises any of standard pharmaceutically accepted carriers known to those of ordinary skill in the art in formulating pharmaceutical compositions. Thus, the compounds, by themselves, such as being present as pharmaceutically acceptable salts, or as conjugates, may be prepared as formulations in pharmaceutically acceptable diluents; for example, saline, phosphate buffer saline (PBS), aqueous ethanol, or solutions of glucose, mannitol, dextran, propylene glycol, oils (e.g., vegetable oils, animal oils, synthetic oils, etc.), microcrystalline cellulose, carboxymethyl cellulose, hydroxylpropyl methyl cellulose, magnesium stearate, calcium phosphate, gelatin, polysorbate 80 or the like, or as solid formulations in appropriate excipients.

The pharmaceutical compositions will often further comprise one or more buffers (e.g., neutral buffered saline or phosphate buffered saline), carbohydrates (e.g., glucose, mannose, sucrose or dextrans), mannitol, proteins, polypeptides or amino acids such as glycine, antioxidants (e.g., ascorbic acid, sodium metabisulfite, butylated hydroxytoluene, butylated hydroxyanisole, etc.), bacteriostats, chelating agents such as EDTA or glutathione, solutes that render the formulation isotonic, hypotonic or weakly hypertonic with the blood of a recipient, suspending agents, thickening agents, preservatives, flavoring agents, sweetening agents, and coloring compounds as appropriate.

The pharmaceutical compositions of the invention are administered in a manner compatible with the dosage formulation, and in such amount as will be therapeutically effective. The quantity to be administered depends on a variety of factors including, e.g., the age, body weight, physical activity, and diet of the individual, the condition or disease to be treated, and the stage or severity of the condition or disease. In certain embodiments, the size of the dose may also be determined by the existence, nature, and extent of any adverse side effects that accompany the administration of a therapeutic agent(s) in a particular individual.

It will be understood, however, that the specific dose level and frequency of dosage for any particular patient may be varied and will depend upon a variety of factors including the activity of the specific compound employed, the metabolic stability and length of action of that compound, the age, body weight, hereditary characteristics, general health, sex, diet, mode and time of administration, rate of excretion, drug combination, the severity of the particular condition, and the host undergoing therapy.

In certain embodiments, the dose of the compound may take the form of solid, semi-solid, lyophilized powder, or liquid dosage forms, such as, for example, tablets, pills, pellets, capsules, powders, solutions, suspensions, emulsions, suppositories, retention enemas, creams, ointments, lotions, gels, aerosols, foams, or the like, preferably in unit dosage forms suitable for simple administration of precise dosages.

As used herein, the term “unit dosage form” refers to physically discrete units suitable as unitary dosages for humans and other mammals, each unit containing a predetermined quantity of a therapeutic agent calculated to produce the desired onset, tolerability, and/or therapeutic effects, in association with a suitable pharmaceutical excipient (e.g., an ampoule). In addition, more concentrated dosage forms may be prepared, from which the more dilute unit dosage forms may then be produced. The more concentrated dosage forms thus will contain substantially more than, e.g., at least 1, 2, 3, 4, 5, 6, 7, 8, 9, 10, or more times the amount of the therapeutic compound.

In particular embodiments, a small molecule compound, e.g,. JZL-184 or (R)-3t, is administered at a dose of about 0.1-100 mg/kg, about 1-50 mg/kg, about 1-20 mg/kg, or about 1, 2, 3, 4, 5, 10, 15, 20, 25, 30, 35, 40, 45, or 50 mg/kg. In some embodiments, e.g., when a MAGL inhibitor, e.g., a small molecule inhibitor, is administered with an opioid in a single pharmaceutical composition, the small molecule or other inhibitor of MAGL can be present ata molar ratio of, e.g., about 1:1000, 1:500, 1:100, 1:90, 1:80, 1:70, 1:60, 1:50, 1:40, 1:30, 1:20, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 20:1, 30:1, 40:1, 50:1, 60:1, 70:1, 80:1, 90:1, 100:1, 500:1, or 1000:1, relative to the opioid. In any of the herein embodiments, an opioid administered together with or in parallel to (e.g., the MAGL inhibitor is administered chronically over the same time period as the opioid, even if according to independent administration regimens) a MAGL inhibitor can be formulated or administered according to any dose, e.g., (e.g., 1-100 MME/day, 10-90 MME/day, 5, 10, 15, 20, 25, 30, 35, 40, 45, 50, 55, 60, 65, 70, 75, 80, 85, 90 MME/day), and in any form (including rapid onset, extended release, short-acting and long acting forms, tablets, capsules, injectable suspensions, injectable solutions, suppositories, parenteral solutions, transdermal patches).

Methods for preparing such dosage forms are known to those skilled in the art (see, e.g., REMINGTON'S PHARMACEUTICAL SCIENCES, supra). The dosage forms typically include a conventional pharmaceutical carrier or excipient and may additionally include other medicinal agents, carriers, adjuvants, diluents, tissue permeation enhancers, solubilizers, and the like. Appropriate excipients can be tailored to the particular dosage form and route of administration by methods well known in the art (see, e.g., REMINGTON'S PHARMACEUTICAL SCIENCES, supra).

Examples of suitable excipients include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline, syrup, methylcellulose, ethylcellulose, hydroxypropylmethylcellulose, and polyacrylic acids such as Carbopols, e.g., Carbopol 941, Carbopol 980, Carbopol 981, etc. The dosage forms can additionally include lubricating agents such as talc, magnesium stearate, and mineral oil; wetting agents; emulsifying agents; suspending agents; preserving agents such as methyl-, ethyl-, and propyl-hydroxy-benzoates (i.e., the parabens); pH adjusting agents such as inorganic and organic acids and bases; sweetening agents; and flavoring agents. The dosage forms may also comprise biodegradable polymer beads, dextran, and cyclodextrin inclusion complexes.

For oral administration, the therapeutically effective dose can be in the form of tablets, capsules, emulsions, suspensions, solutions, syrups, sprays, lozenges, powders, and sustained-release formulations. Suitable excipients for oral administration include pharmaceutical grades of mannitol, lactose, starch, magnesium stearate, sodium saccharine, talcum, cellulose, glucose, gelatin, sucrose, magnesium carbonate, and the like.

The therapeutically effective dose can also be provided in a lyophilized form. Such dosage forms may include a buffer, e.g., bicarbonate, for reconstitution prior to administration, or the buffer may be included in the lyophilized dosage form for reconstitution with, e.g., water. The lyophilized dosage form may further comprise a suitable vasoconstrictor, e.g., epinephrine. The lyophilized dosage form can be provided in a syringe, optionally packaged in combination with the buffer for reconstitution, such that the reconstituted dosage form can be immediately administered to an individual.

In some embodiments, additional compounds or medications can be co-administered to the subject. Such compounds or medications can be co-administered for the purpose of alleviating signs or symptoms of the disease being treated, reducing side effects caused by induction of the immune response, etc. In some embodiments, for example, the MAGL inhibitors of the invention are administered together with an opioid, with another analgesic compound, with a CB1 and/or CB2 agonist, and/or with any other compound potentially attenuating reward and/or delaying tolerance to the analgesic properties of opioids.

### Kits

Other embodiments of the compositions described herein are kits comprising a MAGL inhibitor. The kit typically contains containers, which may be formed from a variety of materials such as glass or plastic, and can include for example, bottles, vials, syringes, and test tubes. A label typically accompanies the kit, and includes any writing or recorded material, which may be electronic or computer readable form providing instructions or other information for use of the kit contents.

In some embodiments, the kit comprises one or more reagents for the treatment of a subject undergoing opioid-based treatment for analgesic purposes. In some embodiments, the kit comprises an agent that antagonizes the expression or activity of MAGL. In some embodiments, the kit comprises an inhibitory nucleic acid (e.g., an antisense RNA, small interfering RNA (siRNA), microRNA (miRNA), short hairpin RNA (shRNA)), or a polynucleotide encoding a MAGL inhibiting polypeptide, that inhibits or suppresses MAGL mRNA or protein expression or activity, e.g., enzyme activity. In some embodiments, the kit comprises a modified RNA, e.g., a modified shRNA or siRNA, or a modified mRNA encoding a polypeptide MAGL inhibitor. In some embodiments, the kit further comprises one or more plasmid, bacterial or viral vectors for expression of the inhibitory nucleic acid or polynucleotide encoding a MAGL-inhibiting polypeptide. In some embodiments, the kit comprises an antisense oligonucleotide capable of hybridizing to a portion of a MAGL-encoding mRNA. In some embodiments, the kit comprises an antibody (e.g., a monoclonal, polyclonal, humanized, bispecific, chimeric, blocking or neutralizing antibody) or antibody-binding fragment thereof that specifically binds to and inhibits a MAGL protein. In some embodiments, the kit comprises a blocking peptide. In some embodiments, the kit comprises an aptamer (e.g., a peptide or nucleic acid aptamer). In some embodiments, the kit comprises an affimer. In some embodiments, the kit comprises a modified RNA. In particular embodiments, the kit comprises a small molecule inhibitor, e.g., JZL-184, (R)-3t or ABX-1431, that binds to MAGL or inhibits its enzymatic activity. In some embodiments, the kit further comprises one or more additional therapeutic agents, e.g., an opioid such as morphine, codeine, fentanyl, hydrocodone, hydromorphone, meperidine, methadone, or oxycodone.

In some embodiments, the kits can further comprise instructional materials containing directions (i.e., protocols) for the practice of the methods of this invention (e.g., instructions for using the kit for attenuating the rewarding effects of and/or delaying tolerance to the analgesic properties of an opioid. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to interne sites that provide such instructional materials.

### EXAMPLES

The following examples are offered to illustrate, but not to limit, the claimed invention.

### Example 1. MAGL Inhibition with JZL-184 Abolishes Morphine's Rewarding Effects While Preserving Morphine's Analgesic Effects and Delays Morphine Induced-Tolerance of its Analgesic Effects

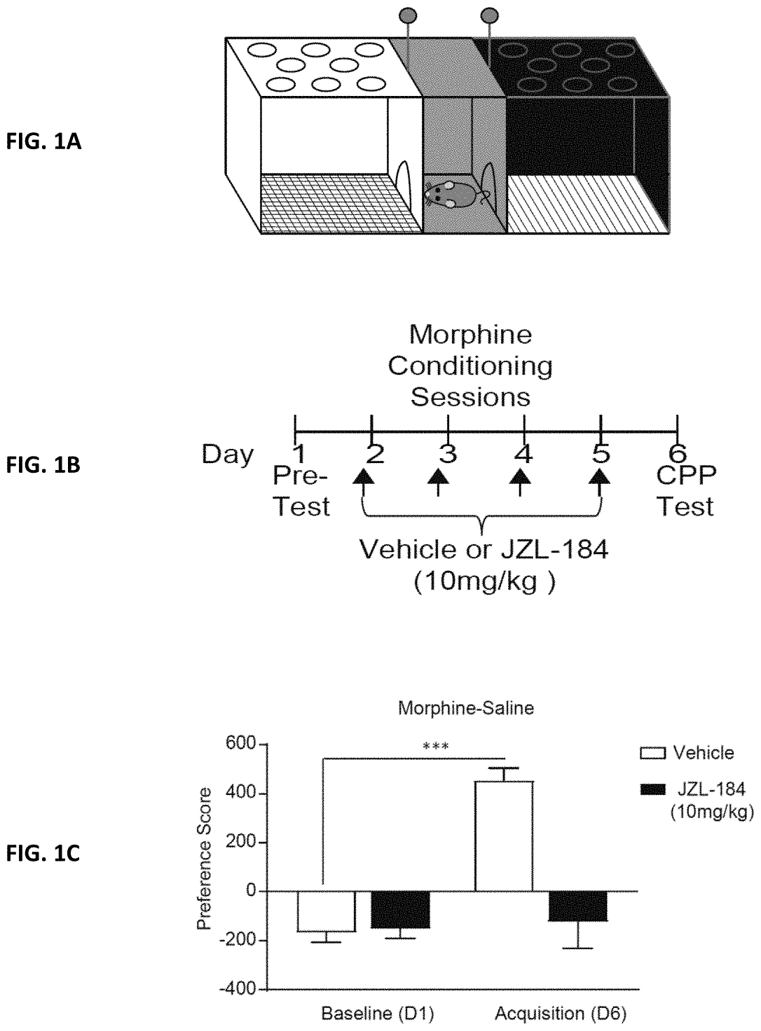

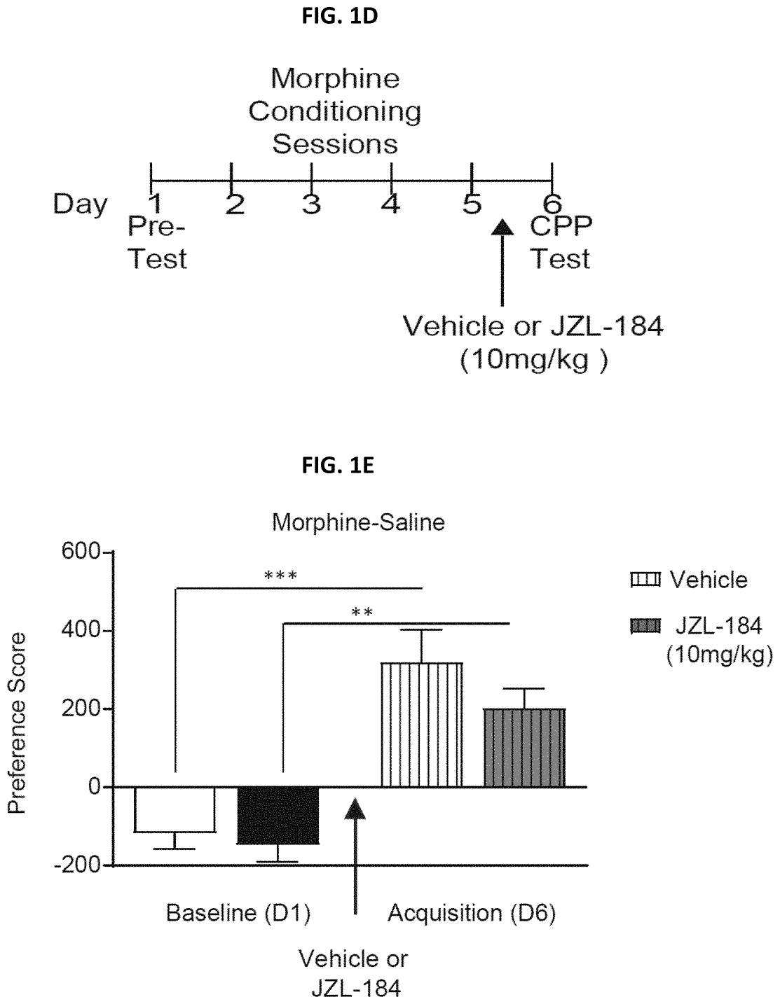

Endocannabinoid signaling is primarily regulated by MAGL and FAAH, the two enzymes responsible for catabolizing the endogenous cannabinoids, 2-AG and anandamide. Although some studies have shown that inhibition of these enzymes reduces certain aspects of morphine withdrawal, no studies exist that have examined their role in morphine reward. We utilized the morphine conditioned place preference (CPP) paradigm, to assess morphine reward, in which mice are conditioned to associate a particular chamber with the rewarding effects of morphine (FIG. 1A). Once conditioned, mice demonstrate a preference for the morphine-paired chamber, demonstrating morphine reward. To test the effect of systemic blockade of MAGL with the pharmacological inhibitor JZL-184 on morphine reward, on Day 1 mice underwent a baseline test, on Day 2-5 mice were conditioned to morphine wherein they received an intraperitoneal (i.p.) injection of JLZ-184 (10 mg/kg, i.p.), two hours prior to each morphine conditioning session (FIG. 1B). On day 6, mice were tested for morphine preference. JZL-184 treatment during morphine conditioning abolished morphine reward, as seen by the lack of morphine place preference (FIG. 1C). Next, to test if this effect of MAGL inhibition was selective for the conditioning phase or potentially could also alter the expression of the morphine preference, mice were injected with JLZ-184 two hours prior to the Day 6 morphine preference test, in the absence of morphine (FIG. 1D). Interestingly, pharmacological inhibition of MAGL before the expression test had no effect on morphine reward (FIG. 1E), demonstrating that MAGL is involved in mechanisms required for establishing morphine reward.

Next, to test the effect of JZL-184 on morphine analgesia, we utilized the tail flick test (FIG. 2A), used to test pain response in animals, in a cumulative dose response of morphine (0.1, 0.3, 1, 3 and 10 mg/kg). Mice were administered JZL-184 (10 mg/kg, i.p.) and two hours later mice received an injection of the lowest dose of morphine (0.1 mg/kg). The antinociception effects of this dose were measured 30 minutes after morphine treatment. The next morphine dose (1 mg/kg, i.p.) was injected immediately after the test, and animals were re-tested in the tail flick test 30 minutes later. This was repeated for the remaining morphine doses (0.3, 3 and 10 mg/kg, i.p.). In contrast to the effect of JZL-184 on morphine reward, inhibiting MAGL did not alter the analgesic effects of morphine (FIG. 2B).

Chronic morphine treatment can result in tolerance to its analgesic effects; therefore, to test if morphine tolerance was affected by MAGL inhibition, for four consecutive days mice received a morning and afternoon injection of JZL-184 (10 mg/kg, i.p.), two hours prior to an injection of morphine (10 mg/kg), and antinociception was assessed every other day in the tail flick test. JZL-184 delayed morphine-induced tolerance compared to that seen in vehicle-treated mice (FIG. 2C).

### Example 2. MAGL Inhibition with Reversible MAGL Inhibitor Abolishes Morphine's Rewarding Effects

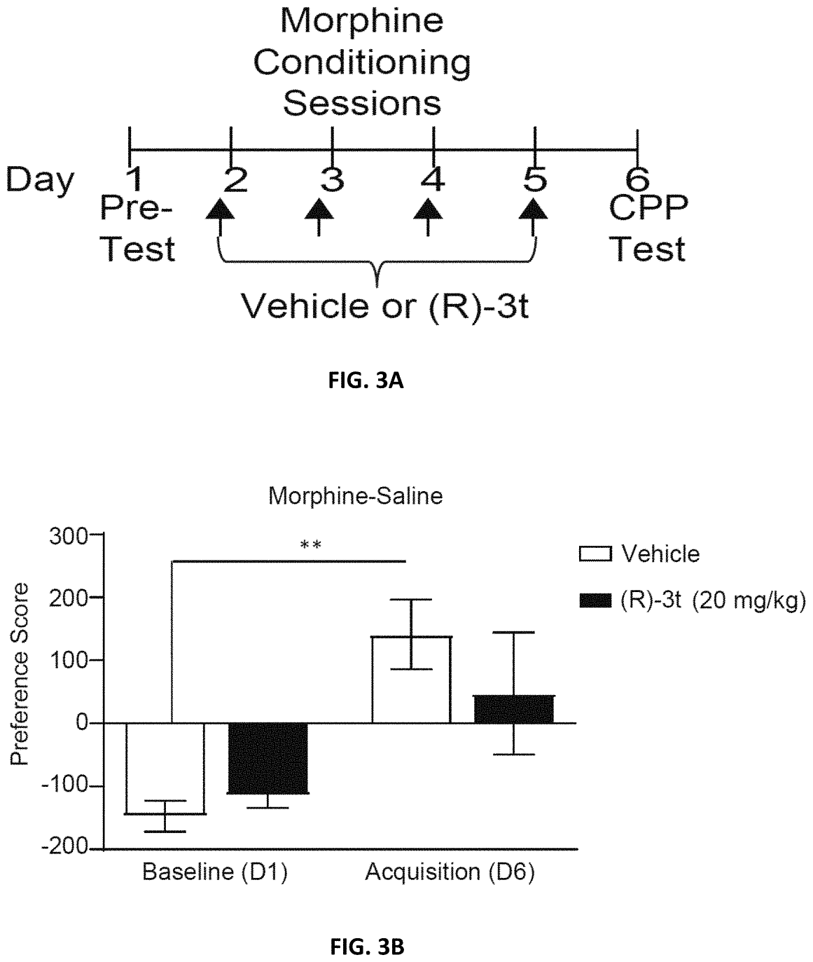

We reported that JZL-184, an irreversible MAGL inhibitor, abolishes morphine reward. In order to expand our findings, we decided to test the (R)-3t compound, a reversible MAGL inhibitor, in morphine conditioned place preference. As with JZL-184, (R)-3t (20 mg/kg) was given before each morphine conditioning session (FIG. 3A). Inhibition of the MAGL enzyme using (R)-3t resulted in a lack of morphine reward, as shown in FIG. 3B. Therefore, inhibition of MAGL with a reversible or an irreversible inhibitor abolishes morphine reward.

### Example 3. FAAH Inhibition with PF-3845 Does Not Alter Morphine Reward, Analgesia or Tolerance to Morphine's Analgesic Effects

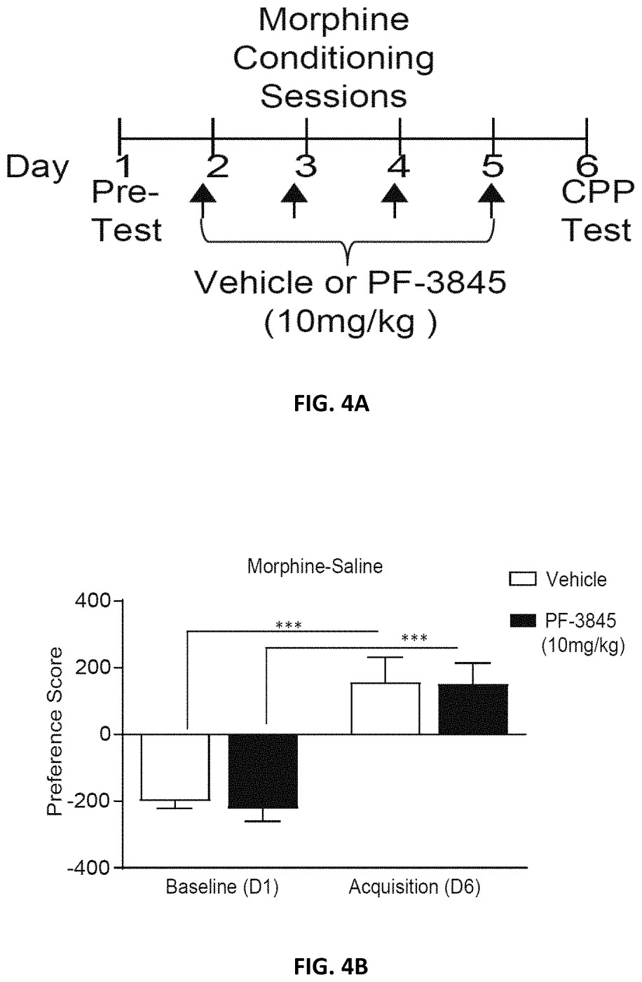

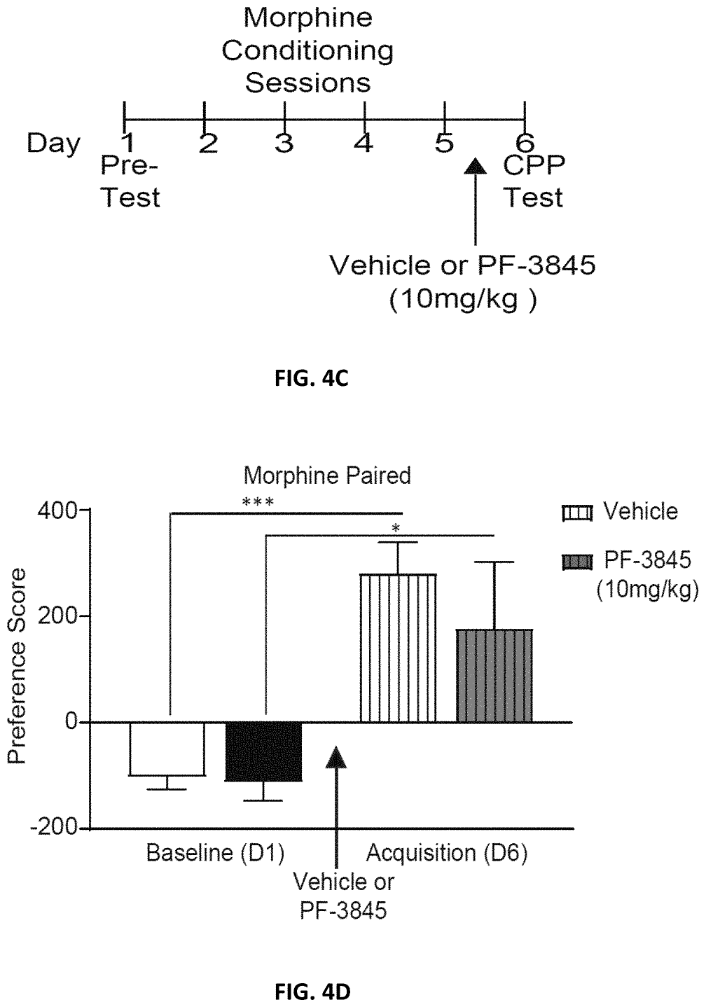

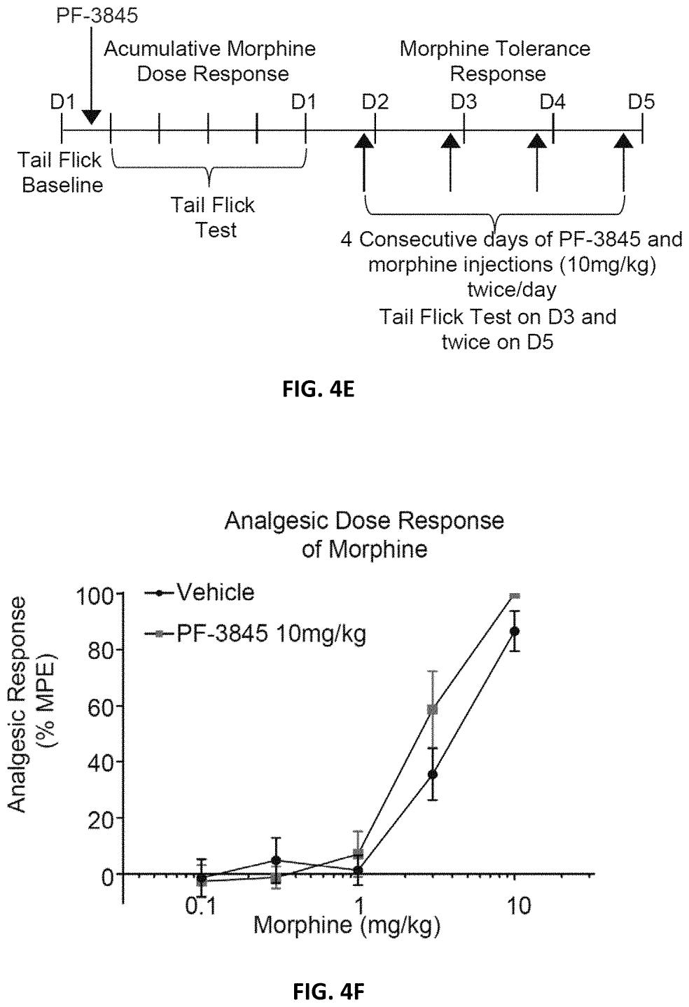

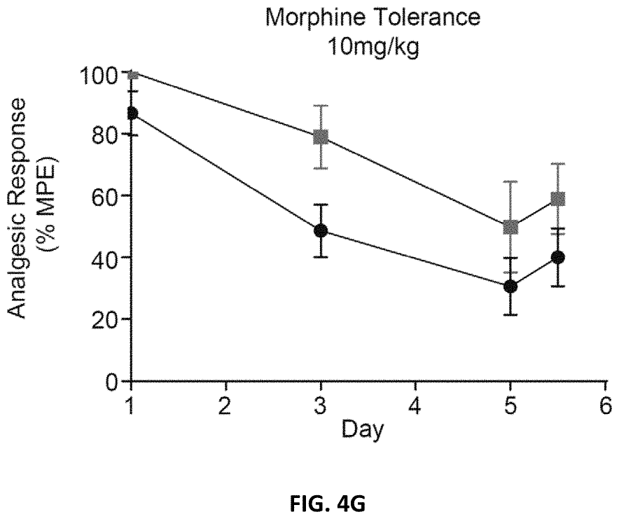

To test the effect of inhibiting the enzyme FAAH on the rewarding effects of morphine, we tested the effect of the FAAH inhibitor PF-3845 in morphine conditioned place preference. In contrast to inhibition of MAGL with JZL-184 and (R)-3t, inhibition of FAAH with PF-3845 (10 mg/kg, i.p.) had no effect on acquisition of morphine conditioned place preference (FIGS. 4A-4B), nor on the expression of morphine preference (FIGS. 4C-4D). These results demonstrate that inhibition of MAGL (with JZL-184 and (R)-3t), but not FAAH (with PF-3845) abolishes the rewarding effects of morphine. We additionally found no effect of PF-3845 (10 mg/kg, i.p.) on morphine-induced analgesia and morphine tolerance using the tail-flick test (FIGS. 4E-G).

### Example 4. Cannabinoid Receptor 1 Activation with the Exogenous Ligand Δ⁹-Tetrahydrocannabinol (THC) Does Not Alter Morphine Reward

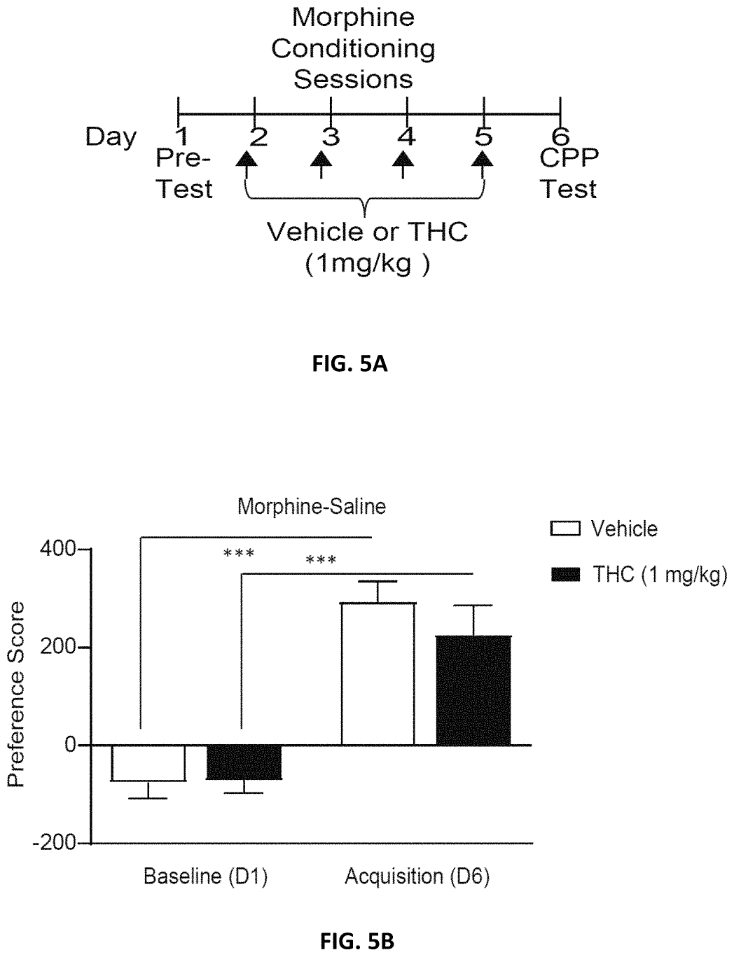

Inhibition of the MAGL enzyme with JZL-184 results in lower metabolization of the endogenous cannabinoid 2-AG. This implies higher availability of this endogenous cannabinoid receptor 1 (CB1) ligand to activate CB1 receptors. Next, we tested the effect of activating the CB1 receptor with the exogenous CB1 ligand, THC, on morphine conditioned place preference. Mice were systemically administered THC prior to each morphine conditioning session (FIG. 5A). THC had no effect on morphine reward as observed by a similar morphine preference in vehicle and THC-pretreated mice (FIG. 5B). This demonstrates that the exogenous CB1 ligand THC is not effective in counteracting morphine's rewarding effects.

### Example 5. MAGL Inhibition Does Not Alter Cocaine CPP

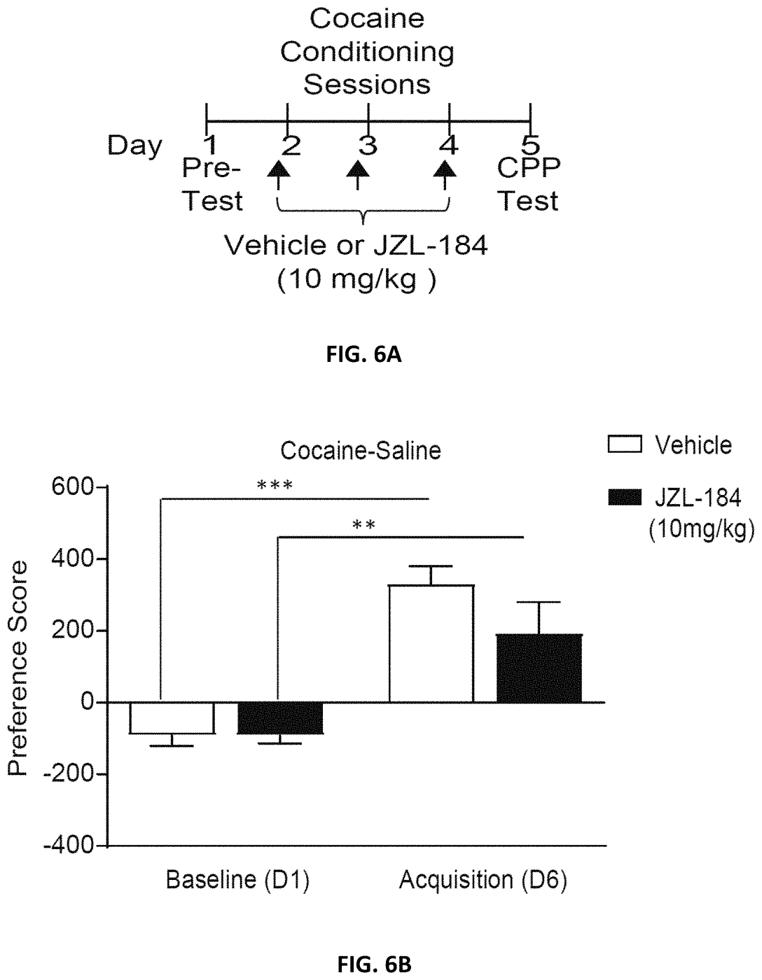

Although drugs of abuse exert their rewarding effects through similar pathways and brain structures, they use different mechanisms. In FIG. 1, we demonstrated the effectiveness of JZL-184 in abolishing morphine reward; therefore, we next tested if JZL-184 was also effective in counteracting cocaine reward. Mice were pretreated with JZL-184 prior to each cocaine conditioning session (FIG. 6A). As opposed to our findings with morphine reward, pharmacological inhibition of MAGL with JZL-184 did not have any effect on cocaine reward (FIG. 6B). This results shows that JZL-184′s effect on reward is drug-specific.

### Example 6. JZL-184 Reduces Nucleus Accumbens Activity Time-Locked to Morphine Conditioned Place Preference Behavior

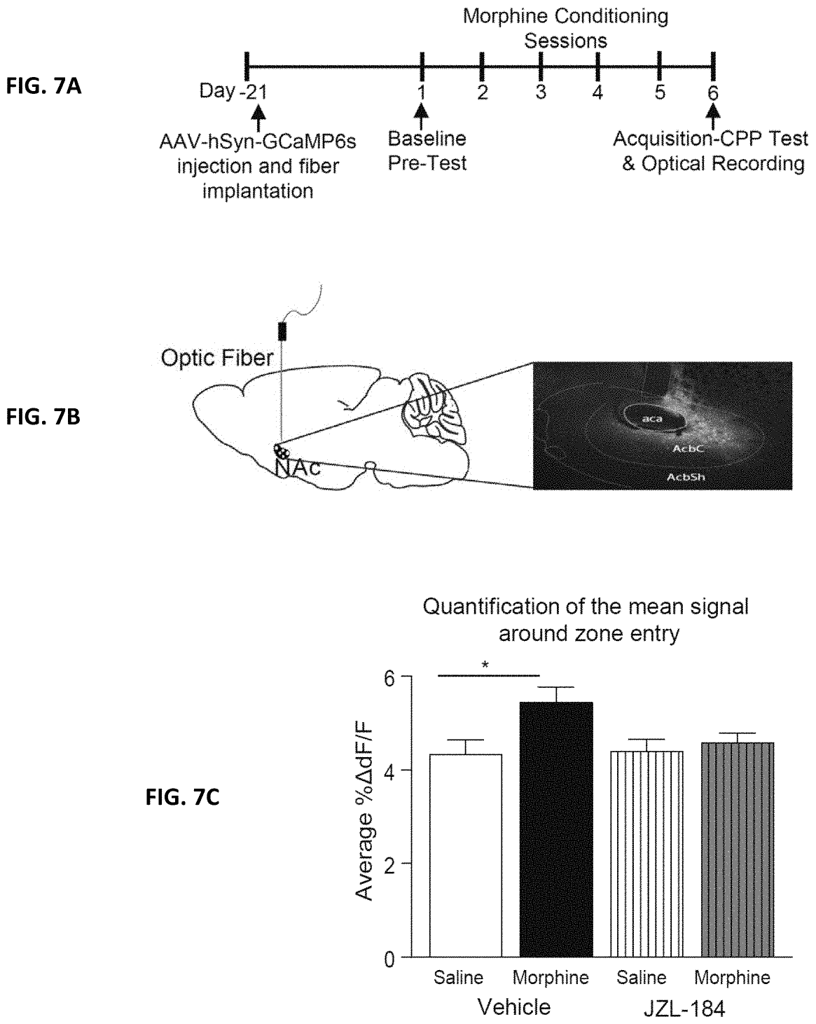

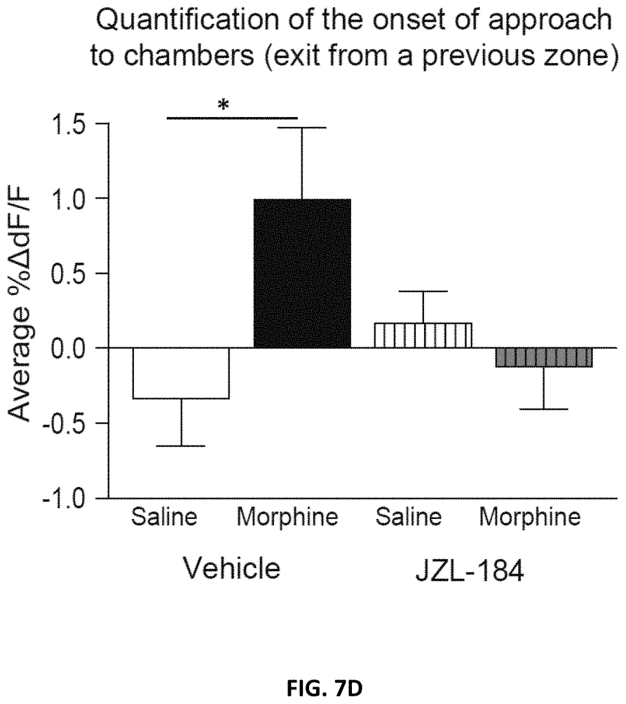

The rewarding effects of morphine have been linked to neural activity of the brain region the nucleus accumbens. To test the effect of JZL-184 on nucleus accumbens activity that underlies morphine's rewarding effects, we used genetically encoded indicator GCaMP6s and in vivo fiber photometry to optically record nucleus accumbens activity dynamics during morphine conditioned place preference. An adeno-associated virus (AAV) expressing GCaMP6s was injected unilaterally into the nucleus accumbens, and an optic fiber was implanted above the injection site to record Ca²⁺ signals, a proxy for neural activity (FIGS. 7A-7B). Mice were tested in morphine conditioned place preference behavior in the presence or absence of JZL-184. Fiber photometry calcium imaging revealed higher nucleus accumbens activity 5 seconds prior to entry into the morphine-paired chamber compared to activity while entering the saline-paired chamber. This increase in activity was absent in mice treated with JZL-184 (FIG. 7C). A similar result was obtained when we examined neural activity when the animals were approaching the morphine-paired chamber. Control animals showed significantly higher nucleus accumbens activity when approaching the morphine-paired chamber that was abolished in mice treated with JZL-184 (FIG. 7D).

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

1. A method of attenuating the rewarding effect of an opioid in a subject, the method comprising administering a therapeutically effective amount of a monoacylglycerol lipase (MAGL) inhibitor to the subject.

2. The method of claim 1, wherein the MAGL inhibitor does not substantially decrease the analgesic properties of the opioid in the subject.

3. The method of claim 1, wherein the MAGL inhibitor delays tolerance to the analgesic properties of the opioid in the subject.

4. The method of claim 1, wherein the MAGL inhibitor increases the level of 2-arachidonoylglycerol (2-AG) and/or decreases the level of arachidonic acid (AA) in the subject.

5. The method of claim 1, wherein the MAGL inhibitor is co-administered to the subject with the opioid.

6. The method of claim 1, wherein the MAGL inhibitor is administered to the subject prior to the administration of the opioid.

7. The method of claim 1, wherein the opioid is selected from the group consisting of morphine, codeine, fentanyl, hydrocodone, hydromorphone, meperidine, methadone, tramadol, buprenorphine, and oxycodone.

8. The method of claim 1, wherein the MAGL inhibitor is a small molecule inhibitor.

9. The method of claim 1, wherein the MAGL inhibitor is a reversible inhibitor.

10. The method of claim 1, wherein the MAGL inhibitor is an irreversible inhibitor.

11. The method of claim 8, wherein the MAGL inhibitor is an O-aryl-carbamate and/or benzodioxole compound.

12. The method of claim 11, wherein the O-aryl-carbamate and/or benzodioxole compound is JZL-184.

13. The method of claim 12, wherein the JZL-184 is administered at a dose of about 10 mg/kg.

14. The method of claim 8, wherein the MAGL inhibitor is ABX-1431.

15. The method of claim 8, wherein the MAGL inhibitor is a piperazinyl pyrrolidin-2-one compound.

16. The method of claim 15, wherein the piperazinyl pyrrolidin-2-one compound is (R)-3t.

17. The method of claim 16, wherein the (R)-3t is administered at a dose of about 20 mg/kg.

18. The method of claim 1, wherein the MAGL inhibitor decreases the expression, stability, or activity of MAGL.

19. The method of claim 18, wherein the MAGL inhibitor decreases the enzymatic activity of MAGL.

20. The method of claim 1, wherein the MAGL inhibitor does not substantially inhibit fatty acid amide hydrolase (FAAH).

21. The method of claim 1, wherein the MAGL inhibitor reduces or prevents the activation of the nucleus accumbens during morphine-induced conditioned place preference in an animal model.

22. The method of claim 1, wherein the subject is a human.

23. The method of claim 1, wherein the subject has an acute or chronic pain condition selected from the group consisting of dental pain, postsurgical pain, musculoskeletal pain, trauma-associated pain, cancer-associated pain, palliative care associated pain, abdominal pain, pelvic pain, infection-associated pain, nephrolithiasis-associated pain, headaches, neuropathic pain, arthritis-associated pain, and cholecystitis-associated pain.

24. The method of claim 1, wherein the subject has depression, post-traumatic stress disorder, or an anxiety disorder.

25. The method of claim 24, wherein the subject is being administered an anti-depressant that acts at least in part through an opioid receptor.

26. The method of claim 22, wherein the subject is an adult or an adolescent.

27. The method of claim 1, wherein the MAGL inhibitor is administered intravenously, intracranially, intracerebroventricularly, intrathecally, intraspinally, intraperitoneally, intramuscularly, intralesionally, intranasally, orally, or subcutaneously.

28. The method of claim 27, wherein the MAGL inhibitor is administered intraperitoneally.

29. The method of claim 1, wherein the MAGL inhibitor is administered once or twice per day.

30-43. (canceled)
